(19) United States
(12) Patent Application Publication
(10) Pub. No.: US 2004/0092584 A1
(43) Pub. Date: May 13, 2004

(54) Substance or composition for the treatment of cancer

(76) Inventors: Johannes Christiaan Swarts, Dan Pienaar (ZA); Constance Elizabeth Medlen, Rietondale (ZA)

Correspondence Address: ROBERT D. FISH; RUTAN & TUCKER, LLP, P.O. BOX 1950, 611 ANTON BLVD., 14TH FLOOR, COSTA MESA, CA, 92628-1950, US

(21) Appl. No.: 10/433,539

(22) PCT Filed: Nov. 29, 2001
(86) PCT No.: PCT/IB01/02258

(30) Foreign Application Priority Data
Dec. 4, 2000 (ZA) 2000/7167

(51) Int. Cl. A61K 31/28; A61K 31/295

(52) U.S. Cl. 514/492; 514/502

(57) ABSTRACT

A substance for use in the treatment of cancer, includes a compound selected from metallocenyl β-diketones Mc-CO—CZ₁Z₂-CO—R. Mc is Fc (ferrocenyl), Rc (ruthenocenyl) or Oc (osmocenyl), R is 11, alkyl or aryl and Z₁ and Z₂ are H, alkyl, aryl or substituted alkyl, ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴. M is Rh or Ir, A¹ and A² are cyclic dienes having 6-8 carbons, or linear alkenes having 2-7 carbons. B¹, B², B³ and B⁴ are selected from CO, P(R¹R²R³), P(OR¹)(OR²)(OR³), R⁴ and X in which R¹, R², R³ and R⁴ are independently alkyl, phenyl or ferrocenyl, and X is a halide or a pseudohalide.

THIS INVENTION relates to the treatment of cancer.

In particular, the invention relates to a substance or composition for use in the treatment of cancer, to the use of a substance or composition in the preparation of a medicament for the treatment of cancer, to a method of treating cancer, to a substance or composition for use in a method of sensitising cells to radiation, to the use of a substance or composition in the preparation of a medicament for use in sensitising cells to radiation, to a method of sensitising cells to radiation and to a metallocenyl β diketone.

According to a first aspect of the invention there is provided a substance or composition for use in the treatment of cancer, the substance or composition including at least one compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl or aryl and Z₁ and Z₂ are independently H, alkyl, aryl or substituted alkyl, ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which

M is selected from Rh and Ir,

A¹ and A² are the same or different and are selected from cyclic dienes having 6-8 carbons, or linear alkenes having 2-7 carbons

B¹, B², B³ and B⁴ are the same or different and are selected from CO, P(R¹R² R³), P(OR¹)(OR²)(OR³), R⁴ and X in which R¹, R², R³ and R⁴ are the same or different and are independently selected from alkyl, phenyl and ferrocenyl, and X is a halide or a pseudohalide, and the method including the step of administering to a person or animal in need of treatment a therapeutically effective dose of the substance or composition.

Z₁ and Z₂ may be selected from haloalkyl and benzyl. R may be selected from CF₃, CCl₃, CH₃, H, Ph (phenyl) and Mc. The halide may be F, Cl, Br or I. The pseudohalide may be N₃, NCO or SCN.

The compound may be selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer [M(β-diketonato)(CO)(COR³)(X)], [M(β-diketonato)(CO)(PR²₃)(R³)(X)] or its acyl isomer [M(β-diketonato)(PR²₃)(COR³)(X)], [M (β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

Preferred compounds in accordance with the Invention are set out in the following compound list.

### COMPOUND LIST

(1) ferrocenoylacetaldehyde, (Hfch),

(2) ferrocenoyltrichloroacetone, (Hfctca),

(3) (η⁴-1,5-cyclooctadiene)(1-ferrocenyl-1,3-propanedionato-(4) κ²O,O′)rhodium(1), [Rh(FcH)(cod)],

(4) (η⁴-1,5-cyclooctadiene)(1,3-diferrocenyl-1,3-propanedionato-κ²O,O′)rhodium(1) [Rh(dfcm)(cod)],

(5) (η⁴-1,5-cyclooctadiene)(1-ferrocenyl-3-phenyl-1,3-propanedionato-κ²O,O′)rhodium(1) [Rh(bfcm)(cod)],

(6) (η⁴-1,5-cyclooctadiene)(1-ferrocenyl-1,3-butanedionato-κ²O,O′)rhodium(1), [Rh(fca)(cod)],

(7) (η⁴-1,5-cyclooctadiene)(1-ferrocenyl-4,4,4-trichloro-1,3-butanedionato-κ²O,O′)rhodium(1), [Rh(fctca)(cod)],

(8) (η⁴-1,5-cyclooctadiene)(1-ferrocenyl-4,4,4-trifluooro-1,3-butanedionato-κ²O,O′)rhodium(1), [Rh(fctfa)(cod)],

(9) diferrocenoylmethane, (Hdfcm),

(10) ferrocenoyltrifluoroacetone, (Hfctfa),

(11) (η⁴-1,5-cyclooctadiene)(1-ferrocenyl-4,4,4-trifluoro-1,3-butanedionato-κ²O,O′)iridium(1), [Ir(fctfa)(cod)],

(12) (η⁴-1,5-cyclooctadiene)(1-ferrocenyl-1,3-butanedionato-κ²O,O′)iridium(1), [Ir(fca)(cod)],

(13) (η⁴-1,5-cyclooctadiene)(1-ferrocenyl-3-phenyl-1,3-propanedionato-κ²O,O′)iridium(1), [Ir(bfcm)(cod)],

(14) benzoylferrocenoylmethane, (Hbfcm),

(15) ferrocenoylacetone, (Hfca),

(16) (η⁴-1,5-cyclooctadiene)(1-ferrocenyl-1,3-propanedionato-κ²O,O′)iridium(1), [Ir(fch)(cod)].

(17) (η⁴-1,5-cyclooctadiene)(1-ruthenocenyl-1,3-propanedionato-κ²O,O′)rhodium(1), [Rh(RcH)(cod)],

(18) (η⁴-1,5-cyclooctadiene)(1,3-diruthenocenyl-1,3-propanedionato-κ²O,O′)rhodium(1) [Rh(drcm)(cod)],

(19) (η⁴-1,5-cyclooctadiene)(1-ruthenocenyl-3-phenyl-1,3-propanedionato-κ²O,O′)rhodium(1) [Rh(brcm)(cod)],

(20) (η⁴-1,5-cyclooctadiene)(1-ruthenocenyl-1,3-butanedionato-κ²O,O′)rhodium(1), [Rh(rca)(cod)],

(21) (η⁴-1,5-cyclooctadiene)(1-ruthenocenyl-4,4,4-trichloro-1,3-butanedionato-κ²O,O′)rhodium(1), [Rh(rctca)(cod)],

(22) (η⁴-1,5-cyclooctadiene)(1-ruthenocenyl-4,4,4-trifluooro-1,3-butanedionato-κ²O,O′)rhodium(1), [Rh(rctfa)(cod)],

(23) (η⁴-1,5-cyclooctadiene)(1-osmocenyl-1,3-propanedionato-κ²O,O′)rhodium(1), [Rh(OcH)(cod)],

(24) (η⁴-1,5-cyclooctadiene)(1,3-diosmocenyl-1,3-propanedionato-κ²O,O′)rhodium(1) [Rh(docm)(cod)],

(25) (η⁴-1,5-cyclooctadiene)(1-osmocenyl-3-phenyl-1,3-propanedionato-κ²O,O′)rhodium(1) [Rh(bocm)(cod)],

(26) (η⁴-1,5-cyclooctadiene)(1-osmocenyl-1,3-butanedionato-κ²O,O′)rhodium(1), [Rh(oca)(cod)],

(27) (η⁴-1,5-cyclooctadiene)(1-osmocenyl-4,4,4-trichloro-1,3-butanedionato-κ²O,O′)rhodium(1), [Rh(octca)(cod)],

(28) (η⁴-1,5-cyclooctadiene)(1-osmocenyl-4,4,4-trifluooro-1,3-butanedionato-κ²O,O′)rhodium(1), [Rh(octfa)(cod)],

(29) (η⁴-1,5-cyclooctadiene)(1-ferrocenyl-3-ruthenocenyl-1,3-propanedionato-κ²O,O′)rhodium(1) [Rh(fcrcm)(cod)],

(30) (η⁴-1,5-cyclooctadiene)(1-ferrocenyl-3-osmocenyl-1,3-propanedionato-κ²O,O′)rhodium(1) [Rh(fcocm)(cod)],

(31) (η⁴-1,5-cyclooctadiene)(1-osmocenyl-3-ruthenocenyl-1,3-propanedionato-κ²O,O′)rhodium(1) [Rh(ocrcm)(cod)],

(32) (η⁴-1,5-cyclooctadiene)(1,3-pentanedionato-κ²O,O′)rhodium(1) [Rh(acac)(cod)],

(33) 1-ruthenocenyl-1,3-propanedione=HRcH,

(34) 1,3-diruthenocenyl-1,3-propanedione=Hdrcm,

(35) 1-ruthenocenyl-3-phenyl-1,3-propanedione=Hbrcm,

(36) ruthenocenoylacetone=1-ruthenocenyl-1,3-butanedione=Hrca,

(37) ruthenocenoyltrichloroacetone=1-ruthenocenyl-4,4,4-trichloro-1,3-butanedione=Hrctca,

(38) ruthenocenoyltrifluoroacetone=1-ruthenocenyl-4,4,4-trifluoro-1,3-butanedione=Hrctfa,

(39) 1-osmocenyl-1,3-propanedione=HOcH

(40) 1,3-diosmocenyl-1,3-propanedione=Hdocm

(41) 1-osmocenyl-3-phenyl-1,3-propanedione=Hbocm,

(42) osmocenoylacetone=1-osmocenyl-1,3-butanedione=Hoca,

(43) osmocenoyltrichloroacetone=1-osmocenyl-4,4,4-trichloro-1,3-butanedione=Hoctca,

(44) osmocenoyltrifluoroacetone=1-osmocenyl-4,4,4-trifluoro-1,3-butanedione=Hoctfa,

(45) ferrocenoylosmocenoylmethane=1 ferrocenyl-3-osmocenyl-1,3-propanedione=Hfcocm,

(46) osmocenoylruthenocenoylmethane=1-osmocenyl-3-ruthenocenyl-1,3-propanedione=Hrcocm,

(47) ferrocenoylruthenocenoylmethane=1-ferrocenyl-3-ruthenocenyl-1,3-propanedione=Hfcrcm,

(48) (η⁴-1,5-cyclooctadiene)(1-ferrocenyl-3-ruthenocenyl-1,3-propanedionato-κ²O,O′)rhodium(1) [Rh(fcrcm)(cod)],

(49) (η⁴-1,5-cyclooctadiene)(1-ferrocenyl-3-osmocenyl-1,3-propanedionato-κ²O,O′)rhodium(1) [Rh(fcocm)(cod)]

Examples of compounds in accordance with the invention are set out in the Appendix.

According to another aspect of the invention there is provided the use of a substance or composition in the preparation of a medicament for the treatment of cancer, the substance or composition including at least one compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which

M is selected from Rh and Ir,

A¹ and A² are the same or different and are selected from cyclic dienes having 6-8 carbons or linear alkenes having 2-7 carbons

B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide.

Z₁ and Z₂ may be selected from haloalkyl and benzyl. R may be selected from CF₃, CCl₃, CH₃, H, Ph (phenyl) and Mc. The halide may be selected from F, Cl, Br and I. The pseudohalide may be selected from N₃, NCO and SCN.

The compound may be selected from the group consisting of [M (β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer, [M(β-diketonato)(CO)(COR³)(X)], [M(β-diketonato)(CO)(PR²₃)(R³)(X)] or its acyl isomer, [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

Preferred compounds in accordance with the invention are set out in the Compound List.

According to another aspect of the invention there is provided a method of treating cancer, the method including the step of administering to a person or animal in need of treatment a therapeutically effective dose of a substance or composition which includes a compound selected from metallocenyl β-diketones of the general formula Fc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which

M is selected from Rh and Ir,

A¹ and A² are the same or different and are selected from cyclic dienes having 6-8 carbons or linear alkenes having 2-7 carbons

B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide.

Z₁ and Z₂ may be selected from haloalkyl and benzyl. R may be selected from CF₃, CCl₃, CH₃, H, Ph (phenyl) and Mc. The halide may be F, Cl, Br or I. The pseudohalide may be N₃, NCO or SCN.

The compound may be selected from the group consisting of [M(β-diketonato)(cod)], [M(β-diketonato)(CO)₂], [M(β-diketonato)(CO)(PR²₃), [M(β-diketonato){P—(OR²)₃}₂] [M(β-diketonato)(CO)₂(R³)(X)] or its acyl isomer [M(β-diketonato)(CO)(COR³)(X)], [M(β-diketonato)(CO)(PR²₃)(R³)(X)] or its acyl isomer [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

Preferred compounds in accordance with the invention are set out in the Compound List.

The compounds of the invention are of particular use in the treatment of a patient with cancer which has built up, or could build up, resistance to other therapeutically active substances. The compounds of the invention are also of particular use in the treatment of a patient with cancer which has built up, or could build up, resistance to radiotherapy, and can be administered before, together with or after radiotherapy.

According to another aspect of the invention there is provided a substance or composition for use in a method of sensitising cells to radiation, the substance of composition including a compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which

M is selected from Rh and Ir,

A¹ and A² are the same or different and are selected from cyclic dienes having 6-8 carbons or linear alkenes having 2-7 carbons

B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide.

Z₁ and Z₂ may be selected from haloalkyl and benzyl. R may be selected from CF₃, CCl₃, CH₃, H, Ph (phenyl) and Mc. The halide may be F, Cl, Br or I. The pseudohalide may be N₃, NCO or SCN.

The compound may be selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer [M(β-diketonato)(CO)(COR³)(X)], [M(β-diketonato)(CO)(PR²₃)(R³)(X)] or its acyl isomer [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

Preferred compounds in accordance with the invention are set out in the Compound List.

According to another aspect of the invention there is provided the use of a substance or composition in the preparation of a medicament for use in sensitising cells to radiation, the substance or composition including a compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴in which

M is selected from Rh and Ir,

A¹ and A² are the same or different and are selected from cyclic dienes having 6-8 carbons or linear alkenes having 2-7 carbons

B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide.

Z₁ and Z₂ may be selected from haloalkyl and benzyl. R may be selected from CF₃, CCl₃, CH₃, H, Ph (phenyl) and Mc. The halide may be F, Cl, Br or I. The pseudohalide may be N₃, NCO or SCN.

The compound may be selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer [M(β-diketonato)(CO)(COR³)(X)], [M(β-diketonato)(CO)(PR²₃)(R³)(X)] or its acyl isomer [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

Preferred compounds in accordance with the invention include (η⁴-1,5-cyclooctadiene)(1,3-pentanedionato-κ²O,O′)rhodium(1) [Rh(acac)(cod)], and the compounds in the Compound List.

According to another aspect of the invention there is provided a method of sensitising cells to radiation, the method including the step of exposing the cells before, during or after irradiation, to a compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which

M is selected from Rh and Ir,

A¹ and A² are the same or different and are selected from cyclic dienes having 6-8 carbons or linear alkenes having 2-7 carbons

B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide, and the method including the step of administering to a person or animal in need of treatment a therapeutically effective dose of the substance or composition.

Z₁ and Z₂ may be selected from haloalkyl and benzyl. R may be selected from CF₃, CCl₃, CH₃, H, Ph (phenyl) and Mc. The halide may be F, Cl, Br or I. The pseudohalide may be N₃, NCO or SCN.

The compound may be selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer [M(β-diketonato)(CO)(COR³)(X)], [M(β-diketonato)(CO)(PR²₃)(R³)(X)] or its acyl isomer [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

Preferred compounds in accordance with the invention include (η⁴-1,5-cyclooctadiene)(1,3-pentanedionato-κ²O,O′)rhodium(1) [Rh(acac)(cod)], and the compounds in the Compound List.

The substance or composition will, in particular, be used for sensitizing cells to radiation under hypoxic conditions.

According to another aspect of the invention there is provided a metallocenyl β-diketone of the general formula Mc-CO—CZ₁Z₂-CO—R in which Mc is selected from Fc (ferrocenyl), Rc (ruthenocenyl) and Oc (osmocenyl), R is H, alkyl or aryl and Z₁ and Z₂ are independently H, alkyl, aryl or substituted alkyl, ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which

M is selected from Rh and Ir,

A¹ and A² are the same or different and are selected from cyclic dienes having 6-8 carbons, or linear alkenes having 2-7 carbons

B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide, with the proviso that the compound may not be

(1) ferrocenoyltrichloroacetone, (Hfctca),

(2) (η⁴-1,5-cyclooctadiene)(1,3-diferrocenyl-1,3-propanedionato-κ²O,O′)rhodium(1) [Rh(dfcm)(cod)],

(3) (η⁴-1,5-cyclooctadiene)(1-ferrocenyl-3-phenyl-1,3-propanedionato-κ²O,O′)rhodium(1) [Rh(bfcm)(cod)],

(4) (η⁴-1,5-cyclooctadiene)(1-ferrocenyl-1,3-butanedionato-κ²O,O′)rhodium(1), [Rh(fca)(cod)],

(5) (η⁴-1,5-cyclooctadiene)(1-ferrocenyl-4,4,4-trichloro-1,3-butanedionato-κ²O,O′)rhodium(1), [Rh(fctca)(cod)],

(6) (η⁴-1,5-cyclooctadiene)(1-ferrocenyl-4,4,4-trifluooro-1,3-butanedionato-κ²O,O′)rhodium(1), [Rh(fctfa)(cod)],

(7) diferrocenoylmethane, (Hdfcm),

(8) ferrocenoyltrifluoroacetone, (Hfctfa),

(9) benzoylferrocenoylmethane, (Hbfcm),

(10) ferrocenoylacetone, (Hfca),

(11) (η⁴-1,5-cyclooctadiene)(1,3-pentanedionato-κ²O,O′)rhodium(1) [Rh(acac)(cod)],

Z₁ and Z₂ may be selected from haloalkyl and benzyl. R may be selected from CF₃, CCl₃, CH₃, H, Ph (phenyl) and Mc. The halide may be F, Cl, Br or I. The pseudohalide may be N₃, NCO or SCN.

The compound may be selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer [M(β-diketonato)(CO)(COR³)(X)], [M(β-diketonato)(CO)(PR²₃)(R³)(X)] or its acyl isomer [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

Preferred compounds are set out in the Compound List.

The invention is now described, by way of example, with reference to the accompanying Examples and the Figures in which

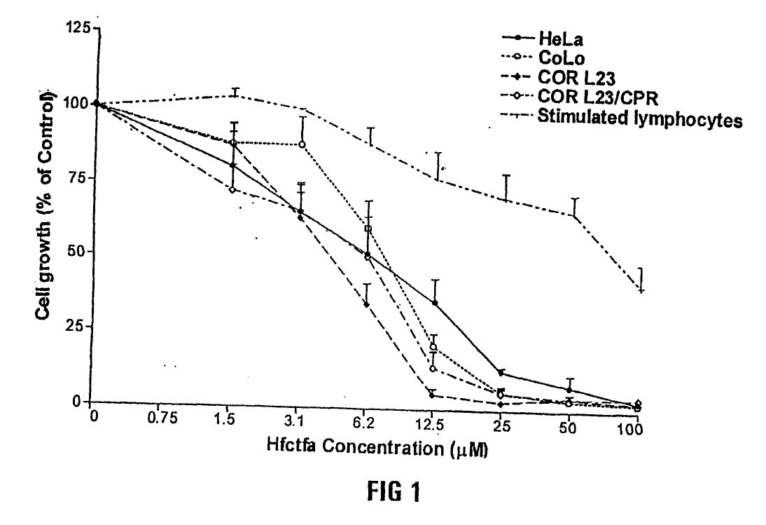

FIG. 1 is a graph showing the growth of various cancer cell lines and PHA-stimulated human lymphocytes as a function of Hfctfa concentration;

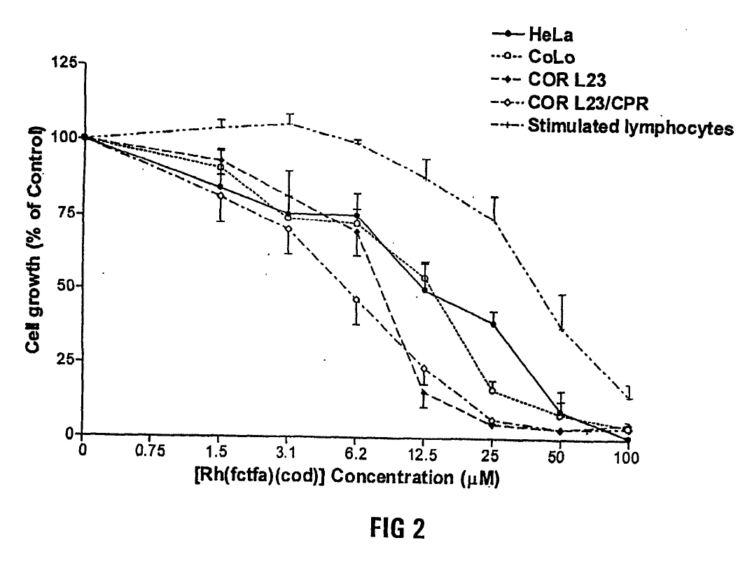

FIG. 2 is a graph of the growth of various cancer cell lines and PHA-stimulated human lymphocites as a function of [Rh(fctfa)(cod)] concentration;

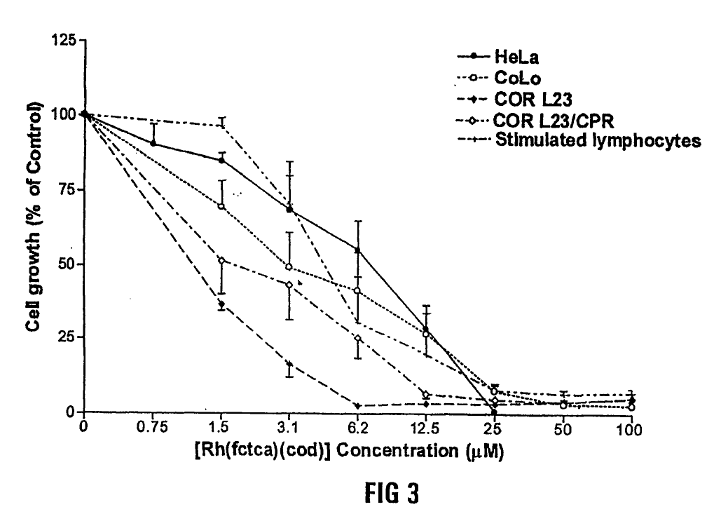

FIG. 3 is a graph of the growth of various cancer cell lines and PHA-stimulated human lymphocites as a function of the concentration of [Rh(fctca)(cod)] concentration;

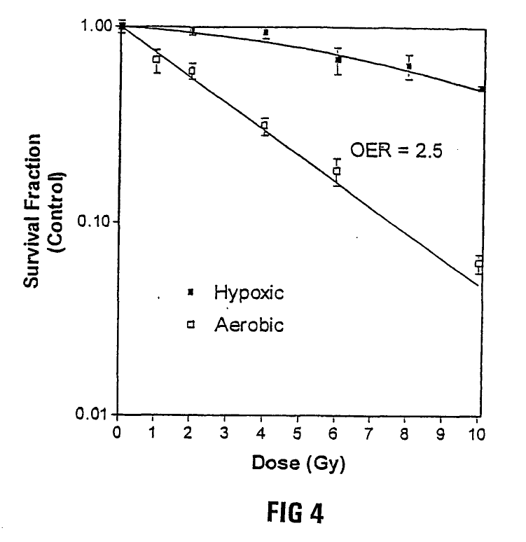

FIG. 4 is a graph of survival fraction as a function of radiation dosage;

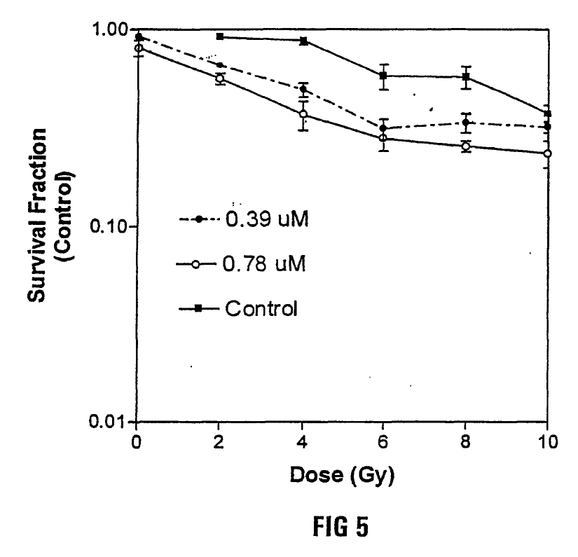

FIG. 5 is a graph of the survival fraction of CHO cells following irradiation under hypoxic conditions in the presence of μM [Rh(fctca)(cod)] as a function of radiation dosage; and

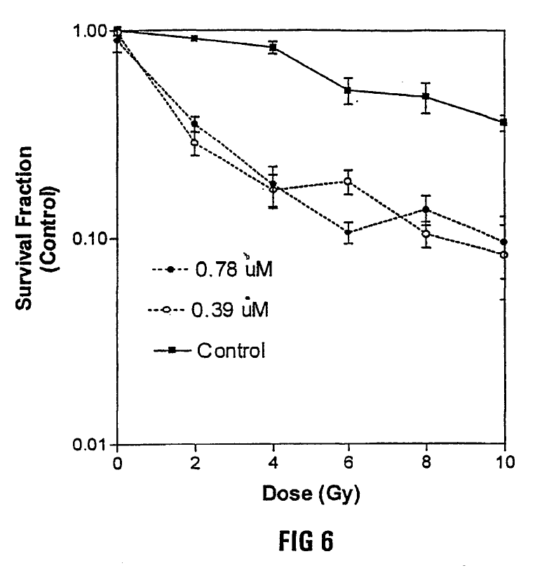

FIG. 6 is a graph of the survival fraction of CHO cells following irradiation under hypoxic conditions in the presence of μM [Rh(fctfa)(cod)] as a function of radiation dosage; and

The examples describe in vitro studies of metallocene-containing β-diketones of the type Mc-CO—CH₂CZ₁Z₂-CO—R in which Mc is as hereinbefore described R is CF₃ (Hfctfa)], CCl₃ (Hfctca), CH₃(Hfca), H (Hfch), Ph (Hbfcm) and Fc (Hdfcm) and their rhodium complexes Rh(β-diketonato)(cod),

### EXAMPLE 1

### Effects of Ferrocene-Containing β-diketones and their Rhodium Complexes on the Growth of Cancer Cell Lines

In these experiments cancer cell lines were cultured in standard tissue culture medium supplemented with 10% fetal calf serum (FCS) at 37° C. In an atmosphere of 5% CO₂ in 96 well round bottom microtitre plates. The cultures were treated with either ferrocene-containing β-diketones or their rhodium complexes at varying concentrations for 72 to 96 h, and the extent of cell growth was assayed by MTT [3-(4,5-dimethylthiazol-2yl)-2,5-diphenyl tetrazolium bromide] reactivity, which detects only viable cells.

The sensitivities of the two cancer cell lines HeLa, which is a sensitive human cervix epitheloid carcinoma cell line, and CoLo 320DM, which is an intrinsically multidrug resistant human colon adeno-carcinoma cell line, to 13 of the complexes of the invention was tested.

Eight of the more active complexes were also tested on the two cell lines COR L23, which is a sensitive human lung large cell carcinoma cell line, and COR L23/CPR, which is a variant of COR L23 and which is resistant to malphalan and other platinum compounds.

All the ferrocene-containing β-diketones, as well as most of their rhodium-cod complexes inhibited the growth of all of the cancer cell lines tested, including those which are drug resistant, at concentrations <100 μM (Table 1). The most active compound amongst the ferrocene complexes was Hfctfa (Table 1, FIG. 1) whereas the two most active rhodium-ferrocene complexes were [Rh(fctfa)(cod)] and [Rh(fctca)(cod)] (Table 1, FIGS. 2 and 3)

TABLE 1
Chemosensitivity of cancer cell lines and PHA-stimulated human
lymphocytes to ferrocene, rhodium-ferrocene complexes and
rhodium ruthenocene complexes
IC 50 (μM)* of the following cell lines for complexes

| | | | | | PHA- |
|---|---|---|---|---|---|
| | | | | | stimulated |
| | | CoLo | COR | COR | lympho- |
| Complexes | HeLa | 320DM | L23 | L23/CPR | cytes |
| Ferrocene: | | | | | |
| Hfch | 73.4 | 80.8 | Nd** | Nd | Nd |
| Hfctca | 37.7 | 28.4 | 12.5 | 20.1 | 67.5 |
| Hdfcm | 54.4 | 64.3 | 75.4 | 74.4 | >100 |
| Hfctfa | 6.8 | 7.3 | 4.5 | 6.3 | 83.1 |
| Hbfcm | 54.2 | 85.1 | 66.8 | 80.4 | >100 |
| Hfca | 66.6 | 57.1 | Nd | Nd | Nd |
| Rhodium- | | | | | |
| Ferrocene: | | | | | |
| Rh(acac)(cod) | 97.3 | >100 | Nd | Nd | Nd |
| Rh(FcH)(cod) | 62.4 | 35.4 | Nd | Nd | Nd |
| Rh(dfcm)(cod) | 18.4 | 70.8 | 19.9 | 30.8 | 42.2 |
| Rh(bfcm)(cod) | 28.3 | 87.1 | 22.5 | 22.0 | 97.2 |
| Rh(fca)(cod) | 64.4 | 56.6 | Nd | Nd | Nd |
| Rh(fctca)(cod) | 7.9 | 3.0 | 1.3 | 2.0 | 4.7 |
| Rh(fctfa)(cod) | 12.5 | 13.4 | 8.5 | 5.8 | 41.6 |
| [Rh(RcH)(cod)] | 52.007 | 68.803 | 33.973 | 39.615 | |
| [Rh(drcm)(cod)] | NT | NT | NT | NT | |
| [Rh(brcm)(cod)] | 16.218 | 20.435 | 15.783 | 14.099 | |
| [Rh(rca)(cod)] | 4.863 | 3 | 0.18 | 2.74 | |
| [Rh(rctca)(cod)] | NT | NT | NT | NT | |
| [Rh(rctfa)(cod)] | 54.742 | 67.438 | 49.207 | 47.993 | |
| [Rh(fcrcm)(cod)] | 17.125 | 29.207 | 10.101 | 20.108 | |

*Data from 3-4 experiments are expressed as the mean drug concentration (μM)-causing 50% cell killing.
**not done

### EXAMPLE 2

### Effects of Ferrocene-Containing β-diketones and their Rhodium Complexes on the Proliferation of Human Lymphocytes

In these experiments suspensions of purified human mononuclear leukocytes were cultured in RPMI medium supplemented with 10% fetal calf serum (FCS) at 37° C. in an atmosphere of 5% CO₂ in 96 well round bottom microtitre plates. To some of the wells a mitogen (phytohaemagglutinin, PHA) was added at a concentration of 2.5 μg/ml. The cultures were treated with either ferrocene-containing β-diketones or their rhodium complexes at varying concentrations for 72 h, and the extent of cell growth was assayed by MTT reactivity.

The ferrocene complexes which were tested inhibited 50% of the growth of PHA-stimulated lymphocyte cultures only at concentrations of 67.5 μM and higher (Table 1). The lymphocyte cultures were, however, more sensitive to the rhodium-ferrocene complexes tested with [Rh(bfcm)(cod)] the least toxic to stimulated lymphocytes (Table 1).

The ideal anti-tumor agent should be an agent with high activity against cancer cells, including drug resistant cells, and low activity against stimulated normal human lymphocytes. Hfctfa possesses the highest tumor specificity and is eight times less active against normal human lymphocytes than tumor cells. This compound would therefore be a possible candidate for the treatment of various cancers, including multidrug resistant cancers. Another possible candidate, [Rh(fctfa)(cod)], is 4-5 times more active against tumor cells than against stimulated human lymphocytes.

### EXAMPLE 3

### Effects of Rhodium-Ferrocene Complexes on the Survival of Chinese Hamster Ovary (CHO) Cells after Irradiation under Aerobic and Hypoxic Conditions

CHO cells were treated in glass test tubes with non-toxic concentrations (0.39 μM and 0.78 μM) of the two rhodium-ferrocene complexes [Rh(fctca)(cod)] and [Rh(fctfa)(cod)] and irradiated with an 8 MV photon beam in a modular incubator chamber with a 2 cm tissue equivalent wax buildup (8MVₘₐₓ) under aerobic and hypoxic conditions. An oxygen enhancement ratio of 2.5 (FIG. 4) was obtained indicating that the method used to establish a hypoxic cellular environment was highly effective.

Neither of the two rhodium complexes tested increased the sensitivity of CHO cells to irradiation in an aerobic environment (results not shown). However, both complexes increased the sensitivity of CHO cells under hypoxic conditions (FIGS. 4 and 5). A dose-modifying factor of 2.9 was obtained under hypoxic conditions for 0.78 μM [Rh(fctfa)(cod)] (Table 2) and 1.9 for 0.39 μM [Rh(fctca)(cod)] (Table 3). The dose-modifying factors were calculated as the ratio of mean inactivation doses [calculated from the respective inactivation parameters obtained from the linear quadratic fit of the cell growth fraction (S)]. Published dose modifying factors (DMFs) measured for cisplatin range from 1.2-2 (Chibber R, Stratford I J, O'Neill P, Sheldon P W, Ahmed I, Lee B. 1985. The interaction between radiation and complexes of cis-Pt (II) and Rh (II): Studied at the molecular level. Int J Radiat Biol 48:513-524; Van De Vaart,. P J M, Klaren, H M, Holland I, Begg A C. 1997. Oral platinum analogue JM216, a radiosensitizer in oxic murine cells. Int J Radiat Biol 72(6):675-683).

These results demonstrate that the two rhodium-ferrocene complexes, [Rh(fctfa)(cod)] and [Rh(fctca)(cod)] compare favorably with cisplatin as inducers of hypoxia selective radiosensitization in rapidly proliferating CHO cells at submicromolar concentrations, suggesting that these complexes may be clinically useful in combination with radiation in the treatment of cancers. Initial tests with Hfctfa and Hfctca were also promising.

TABLE 2
Mean inactivation doses calculated from the response of Chinese
hamster ovary cells following treatment with radiation and/or
different concentrations of [Rh(fctfa)(cod)] under hypoxic conditions.
Dose modifying factors are stated as the ratio of mean inactivation
doses.

| | | Dose |
|---|---|---|
| | Mean Inactivation Dose | Modifying |
| Treatment | (Gy) | Factor (DMF) |
| Radiation (Hypoxia) | 6.44 | |
| Rh(fctfa)(cod) 0.39 μM | 2.41 | 2.6 |
| Rh(fctfa)(cod) 0.78 μM | 2.19 | 2.9 |

TABLE 3
Mean inactivation doses calculated from the response of Chinese
hamster ovary cells following treatment with radiation and/or different
concentrations of [Rh(fctca)(cod)] under hypoxic conditions. Dose
modifying factors are stated as the ratio of mean inactivation doses.

| | Mean Inactivation Dose | Dose Modifying |
|---|---|---|
| Treatment | (Gy) | Factor (DMF) |
| Radiation | 7.16 | |
| (Hypoxia) | | |
| Rh(fctca)(cod) | 3.73 | 1.9 |
| 0.39 μM | | |
| Rh(fctca)(cod) | 4.46 | 1.6 |
| 0.78 μM | | |

### EXAMPLE 4

### Synthesis of the Metallocene Compounds

The synthesis of the new ferrocene-containing, ruthenocene-containing and osmocene-containing betadiketones can all be achieved by Claisen condensation of acetylferrocene, acetylruthenocene or acetylosmocene with the appropriate ester under the influence of lithium diisopropylamide. A selected example of each of these classes of compounds is set out below. Co-ordination with rhodium is also demonstrated

### Betadiketone Synthesis

### The Ferrocene Series: The Synthesis of 1-ferrocenyl-1,3-propanedione, HFcH

To an ice-cool solution of acetylferrocene (2.28 g, 10 mmol) in dry, air free THF (18 ml) was added under nitrogen 5.5 ml of a 2.0 mol dm⁻³ THF solution of lithium diisopropyl amide. Stirring continued for 20 minutes during which time a brick red precipitate formed. A solution of dry, cold methyl formiate (0.6 g, 10 mmol) in THF (1 ml) was then added and stirring continued overnight. After 16 hours, diethyl ether (15 ml) was added to the reaction mixture, the precipitate filtered, washed with ether and air dried. The precipitate was then suspended in 0.5 mol dm⁻³ HCl and extracted with ether, the ether extracts dried with MgSO₄, and the solvent removed to liberate HFcH in 35 yield. The crude product can be crystallised from hexane/ether (1:1).

### The Ruthenocene Series: The Synthesis of 1-ruthenocenyl-1,3-butanedione, Hrca

To an ice-cool solution of acetylruthenocene (0.6 g, 2.2 mmol) in dry, air free THF (4 ml) was added under nitrogen 1.1 ml of a 1.6 mol dm⁻³ THF solution of lithium diisopropyl amide. Stirring continued for 20 minutes during which time a cream precipitate formed. A solution of dry ethyl acetate (x ml, 2.2 mmol) in THF (1 ml) was then added and stirring continued overnight. After 16 hours, diethyl ether (15 ml) was added to the reaction mixture, the precipitate filtered, washed with ether and air dried. The precipitate was then suspended in 0.5 mol dm⁻³ HCl and extracted with ether, the ether extracts dried with MgSO₄, and the solvent removed to liberate Hrca in 30-40% yield. The crude product can be purified further by column chromatography on Kieselgel utilising ether/hexane (3:2) as eluent.

### The Osmocene Series: The Synthesis 1-osmocenyl-4,4,4-trifluoro-1,3-butanedione, Hoctfa

To an ice-cool solution of acetylosmocene (0.25 g, 0.69 mmol) in dry, air free THF (1 ml) was added under nitrogen 0.35 ml of a 2 mol dm⁻³ THF solution of lithium diisopropyl amide. Stirring continued for 20 minutes during which time a cream precipitate formed. A solution of dry ethyl trifluoroacetate (0.1 g, 0.69 mmol) in THF (0.6 ml) was then added and stirring continued overnight. After 16 hours, diethyl ether (10 ml) was added to the reaction mixture, the precipitate filtered, washed with ether and air dried. The precipitate was then suspended in 0.5 mol dm⁻³ HCl and extracted with ether, the ether extracts dried with MgSO₄, and the solvent removed to liberate Hoctfa in 30% yield. The crude product can be crystallised from hexane/ether (1:1).

### The Mixed Metallocene Series: The Synthesis of 1-ferrocenyl-3-ruthenocenylpropanedianato, Hfcrcm

To an ice-cool solution of acetylruthenocene (0.6 g, 2.2 mmol) in dry, air free THF (5 ml) was added under nitrogen 1.1 ml of a 2.0 mol dm⁻³ THF solution of lithium diisopropyl amide. Stirring continued for 20 minutes during which time a cream precipitate formed. A solution of dry methyl ferrocenoate (0.537 g, 2.2 mmol) in THF (1 ml) was then added and stirring continued overnight. After 16 hours, diethyl ether (15 ml) was added to the reaction mixture, the precipitate filtered, washed with ether and air dried. The precipitate was then suspended in 0.5 mol dm⁻³ HCl and extracted with ether, the ether extracts dried with MgSO₄, and the solvent removed to liberate Hfcrcm in 30-40% yield. The crude product can be purified further by column chromatography on Kieselgel utilising ether/hexane (3:2) as eluent.

### Rhodium co-ordination

### The synthesis of (η⁴-1,5-cyclooctadiene)(1-ferrocenyl-1,3-propanedionato-κ²O,O′)rhodium(1), [Rh(FcH)(cod)].

To a stirred solution of [Rh₂Cl₂(cod)₂](0.5 g, 1 mmol) in 6 ml DMF was added 1-ferrocenyl-1,3-propanedione (0.512 g, 2 mmol). After 5 minutes, the crude product was precipitated with an excess of water, filtered and dissolved in ether. The ether solution was washed with water, dried with magnesium sulphate. After solvent removal the residue was crystallised from cold ether/hexane mixtures starting with a ratio of 1:1. Pure crystals of [Rh(FcH)(cod)] were filtered in 65% yield from the mother liquor.

### The Synthesis of (η⁴-1,5-cyclooctadiene)(1-ruthenocenyl-1,3-butanedionato-κ²O,O′)rhodium(1), [Rh(rca)(cod)]

To a stirred solution of [Rh₂Cl₂(cod)₂] (0.5 g, 1 mmol) in 6 ml DMF was added 1-ruthenocenyl-1,3-butanedione (0.631 g, 2 mmol). After 5 minutes, the crude product was precipitated with an excess of water, filtered and dissolved in ether. The ether solution was washed with water, dried with magnesium sulphate and the solvent removed. The crude product was then crystallised from cold ether/hexane mixtures starting with a ratio of 1:1. Pure crystals of [Rh(rca)(cod)] were filtered in 75% yield from the mother liquor.

### The Synthesis of (η⁴-1,5-cyclooctadiene)(1-osmocenyl-4,4,4-trifluoro-1,3-propanedionato-κ²O,O′)rhodium(I), [Rh(octfa)(cod)]

To a stirred solution of [Rh₂Cl₂(cod)₂] (0.5 g, 1 mmol) in 6 ml DMF was added 1-osmocenyl-4,4,4-trifluoro-1,3-butanedione (0.917 g, 2 mmol). After 5 minutes, the crude product was precipitated with an excess of water, filtered and dissolved in ether. The ether solution was washed with water, dried with magnesium sulphate. After solvent removal, the residue was crystallised from cold ether/hexane mixtures starting with a ratio of 1:1. Pure crystals of [Rh(octfa)(cod)] were filtered in 55% yield from the mother liquor.

### The Synthesis of (η⁴-1,5-cyclooctadiene)(1-ferrocenyl-3-ruthenocenyl-1,3-propanedionato-κ²O,O′)rhodium(I), [Rh(fcrcm)(cod)]

To a stirred solution of [Rh₂Cl₂(cod)₂] (0.5 g, 1 mmol) in 6 ml DMF was added 1-ferrocenyl-1,3-propanedione (0.97 g, 2 mmol). After 5 minutes, the crude product was precipitated with an excess of water, filtered and dissolved in ether. The ether solution was washed with water, dried with magnesium sulphate and the crude product crystallised after solvent removal from cold ether/hexane mixtures starting with a ratio of 1:1. Pure crystals of [Rh(fcrcm)(cod)] were obtained in 65% yield from the mother liquor.

### Discussion

Cisplatin is one of the most widely used drugs for the chemotherapy of cancer (Muggia F M. 1991; Introduction: Cisplatin update. Seminars in Oncology 18:1-4). However, this compound has serious side effects including nausea, vomiting and nephrotoxicity (Rosenberg B. 1985; Fundamental studies with cisplatin. Cancer 55:2303-2316) all of which are dose limiting. The search for novel organometallic complexes exhibiting higher antineoplastic activity and decreased side-effects, has stimulated the interest of several investigators and many reports are available on the antineoplastic activity of transitional metal complexes. Among them some rhodium complexes appear to be promising antitumor agents (Bear J L, Gray H B, Rainen L, Chang I-M, Howard R, Serio G, Kimball A P, 1975, Interaction of Rhodium II carboxylates with molecules of biologic importance; Cancer Chemother Rep 59:611-620; Fiamiani V, Ainis 7, Cavallaro A, Piraino P.1990; Antitumor effects of the new rhodium (II) complex: Rh₂(Form)₂(O₂CCF₃)₂(H₂O)₂ (Form=N,N′-di-p-tolylformamidinate; J Chemother 2:319-326. Sartori R, Rencoret G, Rencoret G, Mora A, Perez C, Pastene R, Sariego R, Moya S A. 1997; The novel use of Rh (I) complexes with naphthyridine ligands and poly (oxyethylene) as antitumorals. Anti-Cancer Drugs 6:87-92). Some of these rhodium complexes, despite the heavy-metal character of rhodium, seem to show no nephrotoxicity (Kopf-Maier P.1994. Complexes of metals other than platinum as antitumoral agents. Eur J Clin Pharmacol 47:1-16; Craciunescu D G, Scaria V, Furlani A, Papaionnou A, Iglesias E P, Alonso M P. 1991. Pharmacological and toxicological studies on new Rh (I) organometalic complexes. In Vivo 5:329-332).

The presence of hypoxic cells, resistant to radiotherapy as a consequence of the rapid metabolism of oxygen in tumor tissue, is a limiting factor in the successful treatment of tumors by radiations Sensitization of radioresistant tumors can be achieved by the use of chemical radiosensitizers in combination with conventional radiotherapy.

The Applicant has found that metallocene-containing β-diketones of the type Mc-CO—CH₂—CO—R in which Mc is selected from Fc (ferrocenyl), Rc (ruthenocenyl) and Oc (osmocenyl), and R is CF₃, CCl₃, CH₃, H, Ph (phenyl) or Fc, the enol forms of the β-diketones and rhodium and iridium complexes of the β-diketones previously untested in biological systems inhibit the growth of various cancer cells, including platinum resistant strains and multidrug resistant cancer cells. Furthermore, some of these complexes have also been found to sensitize cells to radiation under hypoxic conditions.

The synthesis of Fc-CO—CH₂—CO—R, in which R is CF₃, CCl₃, CH₃, Ph and Fc, as well as Rh(β-diketonato)(cod) has been described (Du Plessis W C, Vosloo T G, Swarts J C, 1998; β-Diketones containing a ferrocenyl group: synthesis, structural aspects, pKₐ¹ values, group electronegativities and complexation with rhodium(I). Dalton Trans., 2507-2514), The published route to the complexes involves at least five intermediates. The Applicant has also found that none of these intermediates, which include ferrocene itself, acetyl ferrocene, esters of the type R⁴COOMe and R⁴COOEt (R⁴=Fc, CF₃, CCl₃, CH₃, H, Ph or Fc), RhCl₃ and [RhCl(cod)]₂, exhibit any significant anticancer activity. The Applicant has found that the compounds of the invention show better chemotherapeutic and radiosensitisation properties than cisplatin.

### Appendix

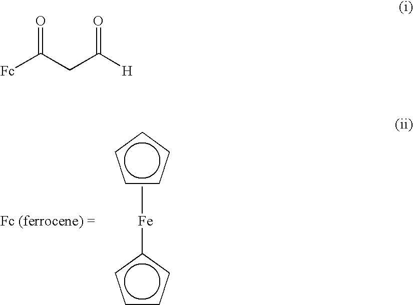

1. (i) The molecular structure of Ferrocenoylacetaldehyde, (Hfch). (ii) Fc refers to ferrocenyl=FeC₁₀H₉═Fe(C₅H₅)(C₅H₄), a dicyclopentadienyl moiety.

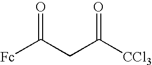

2. The molecular structure of Ferrocenoyltrichloroacetone, (Hfctca). (ii) The molecular structure of cyclooctadiene (cod).

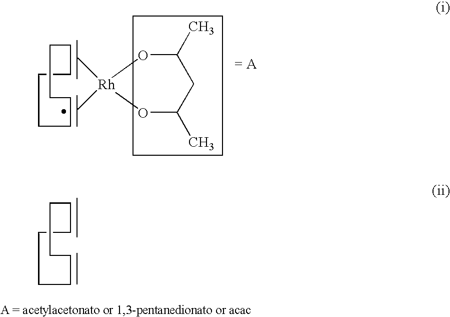

3. (i) The molecular structure of (η⁴-1,5-cyclooctadiene)(1,3-pentanedionato-κ²O,O′)rhodium(1) [Rh(acac)(cod)]. (ii) The molecular structure of cyclooctadiene (cod).

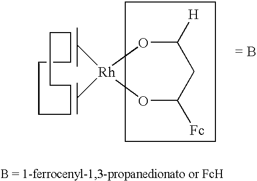

4. The molecular structure of (η⁴-1,5-cyclooctadiene)(1-ferrocenyl-1,3-propanedionato-κ²O,O′)rhodium(1) [Rh(FcH)(cod)].

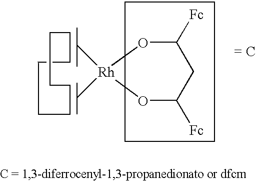

5. The molecular structure of (η⁴-1,5-cyclooctadiene)(1,3-diferrocenyl-1,3-propanedionato-κ²O,O′) rhodium(1) [Rh(dfcm)(cod)].

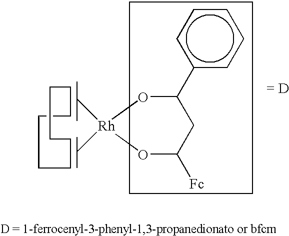

6. The molecular structure of (η⁴-1,5-cyclooctadiene)(1-ferrocenyl-3-phenyl-1,3-propanedionato-κ²O,O′)rhodium(1) [Rh(bfcm)(cod)].

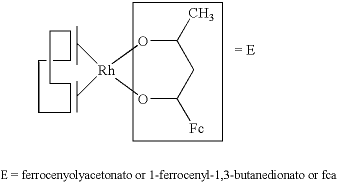

7. The molecular structure of (η⁴-1,5-cyclooctadiene)(1-ferrocenyl-1,3-butanedionato-κ²O,O′)rhodium (1) [Rh(fca)(cod)].

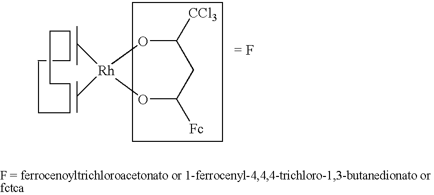

8. The molecular structure of (η⁴-1,5-cyclooctadiene)(1-ferrocenyl-4,4,4-trichloro-1,3-butanedionato-κ²O,O′)rhodium(1) [Rh(fctfa)(cod)].

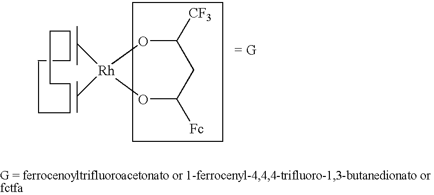

9. The molecular structure of (η⁴-1,5-cyclooctadiene)(1-ferrocenyl-4,4,4-trifluooro-1,3-butanedionato-κ²O,O′)rhodium(1) [Rh(fctfa) (cod)].

10. In all cases Fc in the above structures can be replaced by Rc (ruthenocenyl[Ru(C₅H₅)(C₅H₄)] or Oc (osmocenyl) [Os(C₅H₅)(C₅H₄)].

1. A substance or composition for use in the treatment of cancer, the substance or composition including at least one compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl, ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which
M is selected from Rh and Ir,
A¹ and A² are the same or different and are selected from cyclic dienes having 6-8 carbons or linear alkenes having 2-7 carbons,
B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide, and the method including the step of administering to a person or animal in need of treatment a therapeutically effective dose of the substance or composition.

2. A substance or composition as claimed in claim 1, in which Z₂ and Z₂ are selected from haloalkyl and benzyl.

3. A substance or composition as claimed in claim 1 or claim 2, in which R is selected from CF₃, CCl₃, CH₃, H, Ph (phenyl) and Mc.

4. A substance or composition as claimed in any one of the preceding claims in which the halide is selected from F, Cl, Br and I.

5. A substance or composition as claimed in any one of the preceding claims in which the pseudohalide is selected from N₃, NCO and SCN.

6. A substance or composition as claimed in any one of the preceding claims, in which the compound is selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer, [M(β-diketonato)(CO)(COR³)(X)], [M(β-diketonato)(CO)(PR²₃)(R³)(X)] or its acyl isomer, [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

7. A substance or composition as claimed in any one of the preceding claims, in which the compound is selected from the following compounds 1-49:
(1) ferrocenoylacetaldehyde, (Hfch),
(2) ferrocenoyltrichloroacetone, (Hfctca),
(3) (η⁴-1,5-cyclooctadiene)(1-ferrocenyl-1,3-propanedionato-(4) κ²O,O′)rhodium(1), [Rh(FcH)(cod)],
(4) (η⁴-1,5-cyclooctadiene)(1,3-diferrocenyl-1,3-propanedionato-κ²O,O′)rhodium(1) [Rh(dfcm)(cod)],
(5) (η⁴-1,5-cyclooctadiene)(1-ferrocenyl-3-phenyl-1,3-propanedionato-κ²O,O′)rhodium(1) [Rh(bfcm)(cod)],
(6) (η⁴-1,5-cyclooctadiene)(1-ferrocenyl-1,3-butanedionato-κ²O,O′)rhodium(1), [Rh(fca)(cod)],
(7) (η⁴-1,5-cyclooctadiene)(1-ferrocenyl-4,4,4-trichloro-1,3-butanedionato-κ²O,O′)rhodium [Rh(fctca)(cod)],
(8) (η⁴-1,5-cyclooctadiene)(1-ferrocenyl-4,4,4-trifluooro-1,3-butanedionato-κ²O,O′)rhodium(1), [Rh(fctfa)(cod)],
(9) diferrocenoylmethane, (Hdfcm),
(10) ferrocenoyltrifluoroacetone, (Hfctfa),
(11) (η⁴-1,5-cyclooctadiene)(1-ferrocenyl-4,4,4-trifluoro-1,3-butanedionato-κ²O,O+)iridium(1), [Ir(fctfa)(cod)],
(12) (η⁴-1,5-cyclooctadiene)(1-ferrocenyl-1,3-butanedionato-κ²O,O′)iridium(1), [Ir(fca)(cod)],
(13) (η⁴-1,5-cyclooctadiene)(1-ferrocenyl-3-phenyl-1,3-propanedionato-κ²O,O′)iridium(1), [Ir(bfcm)(cod)],
(14) benzoylferrocenoylmethane, (Hbfcm),
(15) ferrocenoylacetone, (Hfca),
(16) (η⁴-1,5-cyclooctadiene)(1-ferrocenyl-1,3-propanedionato-κ²O,O)iridium(1), [Ir(fch)(cod)].
(17) (η⁴-1,5-cyclooctadiene)(1-ruthenocenyl-1,3-propanedionato-κ²O,O′)rhodium(1), [Rh(RcH)(cod)],
(18) (η⁴-1,5-cyclooctadiene)(1,3-diruthenocenyl-1,3-propanedionato-κ²O,O′)rhodium(1) [Rh(drcm)(cod)],
(19) (η⁴-1,5-cyclooctadiene)(1-ruthenocenyl-3-phenyl-1,3-propanedionato-κ²O,O′)rhodium(1) [Rh(brcm)(cod)],
(20) (η⁴-1,5-cyclooctadiene)(1-ruthenocenyl-1,3-butanedionato-κ²O,O′)rhodium(1), [Rh(rca)(cod)],
(21) (η⁴-1,5-cyclooctadiene)(1-ruthenocenyl-4,4,4-trichloro-1,3-butanedionato-κ²O,O′)rhodium(1), [Rh(rctca)(cod)],
(22) (η⁴-1,5-cyclooctadiene)(1-ruthenocenyl-4,4,4-trifluooro-1,3-butanedionato-κ²O,O′)rhodium(1), [Rh(rctfa)(cod)],
(23) (η⁴-1,5-cyclooctadiene)(1-osmocenyl-1,3-propanedionato-κ²O,O′)rhodium(1), [Rh(OcH)(cod)],
(24) (η⁴-1,5-cyclooctadiene)(1,3-diosmocenyl-1,3-propanedionato-κ²O,O′)rhodium(1) [Rh(docm)(cod)],
(25) (η⁴-1,5-cyclooctadiene)(1-osmocenyl-3-phenyl-1,3-propanedionato-κ²O,O′)rhodium(1) [Rh(bocm)(cod)],
(26) (η⁴-1,5-cyclooctadiene)(1-osmocenyl-1,3-butanedionato-κ²O,O′)rhodium(1), [Rh(oca)(cod)],
(27) (η⁴-1,5-cyclooctadiene)(1-osmocenyl-4,4,4-trichloro-1,3-butanedionato-κ²O,O′)rhodium(1), [Rh(octca)(cod)],
(28) (η⁴-1,5-cyclooctadiene)(1-osmocenyl-4,4,4-trifluooro-1,3-butanedionato-κ²O,O′)rhodium(1), [Rh(octfa)(cod)],
(29) (η⁴-1,5-cyclooctadiene)(1-ferrocenyl-3-ruthenocenyl-1,3-propanedionato-κ²O,O′)rhodium(1) [Rh(fcrcm)(cod)],
(30) (η⁴-1,5-cyclooctadiene)(1-ferrocenyl-3-osmocenyl-1,3-propanedionato-κ²O,O′)rhodium(1) [Rh(fcocm)(cod)],
(31) (η⁴-1,5-cyclooctadiene)(1-osmocenyl-3-ruthenocenyl-1,3-propanedionato-κ²O,O′)rhodium(1) [Rh(ocrcm)(cod)],
(32) (η⁴-1,5-cyclooctadiene)(1,3-pentanedionato-κK²O,O′)rhodium(1) [Rh(acac)(cod)],
(33) 1-ruthenocenyl-1,3-propanedione=HRcH,
(34) 1,3-diruthenocenyl-1,3-propanedione=Hdrcm,
(35) 1-ruthenocenyl-3-phenyl-1,3-propanedione=Hbrcm,
(36) ruthenocenoylacetone=1-ruthenocenyl-1,3-butanedione=Hrca,
(37) ruthenocenoyltrichloroacetone=1-ruthenocenyl-4,4,4-trichloro-1,3-butanedione=Hrctca,
(38) ruthenocenoyltrifluoroacetone=1-ruthenocenyl-4,4,4-trifluoro-1,3-butanedione=Hrctfa,
(39) 1-osmocenyl-1,3-propanedione=HOcH
(40) 1,3-diosmocenyl-1,3-propanedione=Hdocm
(41) 1-osmocenyl-3-phenyl-1,3-propanedione=Hbocm,
(42) osmocenoylacetone=1-osmocenyl-1,3-butanedione=Hoca,
(43) osmocenoyltrichloroacetone=1-osmocenyl-4,4,4-trichloro-1,3-butanedione=Hoctca,
(44) osmocenoyltrifluoroacetone=1-osmocenyl-4,4,4-trifluoro-1,3-butanedione=Hoctfa,
(45) ferrocenoylosmocenoylmethane=1 ferrocenyl-3-osmocenyl-1,3-propanedione=Hfcocm,
(46) osmocenoylruthenocenoylmethane=1-osmocenyl-3-ruthenocenyl-1,3-propanedione=Hrcocm,
(47) ferrocenoylruthenocenoylmethane=1-ferrocenyl-3-ruthenocenyl-1,3-propanedione=Hfcrcm,
(48) (η⁴-1,5-cyclooctadiene)(1-ferrocenyl-3-ruthenocenyl-1,3-propanedionato-κ²O,O′)rhodium(1) [Rh(fcrcm)(cod)],
(49) (η⁴-1,5-cyclooctadiene)(1-ferrocenyl-3-osmocenyl-1,3-propanedionato-κ²O,O′)rhodium(1) [Rh(fcocm)(cod)]

8. Use of a substance or composition in the preparation of a medicament for the treatment of cancer, the substance or composition including at least one compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which
M is selected from Rh and Ir,
A¹ and A² are the same or different and are selected from cyclic dienes having 6-8 carbons or linear alkenes having 2-7 carbons
B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide.

9. Use as claimed in claim 8, in which Z₁ and Z₂ are selected from haloalkyl and benzyl.

10. Use as claimed in claim 8 or claim 9, in which R is selected from CF₃, CCl₃, CH₃, H, Ph (phenyl) and Mc.

11. Use as claimed in any one of claims 8 to 10 inclusive, in which the halide is selected from F, Cl, Br and I.

12. Use as claimed in any one of claims 8 to 11 inclusive, in which the pseudohalide is selected from N₃, NCO and SCN.

13. Use as claimed in any one of claims 8 to 12 inclusive, in which the compound is selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer, [M(β-diketonato)(CO)(COR³)(X)], [M(β-diketonato)(CO)(PR²₃)(R³)(X)] or its acyl isomer, [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

14. Use as claimed in any one of claims 8 to 13 inclusive, in which the compound is selected from the following compounds 1-49:
(1) ferrocenoylacetaldehyde, (Hfch),
(2) ferrocenoyltrichloroacetone, (Hfctca),
(3) (η⁴-1,5-cyclooctadiene)(1-ferrocenyl-1,3-propanedionato-(4) κ²O,O′)rhodium(1), [Rh(FcH)(cod)],
(4) (η⁴-1,5-cyclooctadiene)(1,3-diferrocenyl-1,3-propanedionato-κ²O,O′)rhodium(1) [Rh(dfcm)(cod)],
(5) (η⁴-1,5-cyclooctadiene)(1-ferrocenyl-3-phenyl-1,3-propanedionato-κ²O,O′)rhodium(1) [Rh(bfcm)(cod)],
(6) (η⁴-1,5-cyclooctadiene)(1-ferrocenyl-1,3-butanedionato-κ²O,O′)rhodium(1), [Rh(fca)(cod)],
(7) (η⁴-1,5-cyclooctadiene)(1-ferrocenyl-4,4,4-trichloro-1,3-butanedionato-κ²O,O′)rhodium(1), [Rh(fctca)(cod)],
(8) (η⁴-1,5-cyclooctadiene)(1-ferrocenyl-4,4,4-trifluooro-1,3-butanedionato-κ²O,O′)rhodium(1), [Rh(fctfa)(cod)],
(9) diferrocenoylmethane, (Hdfcm),
(10) ferrocenoyltrifluoroacetone, (Hfctfa),
(11) (η⁴-1,5-cyclooctadiene)(1-ferrocenyl-4,4,4-trifluoro-1,3-butanedionato-κ²O,O′)iridium(1), [Ir(fctfa)(cod)],
(12) (η⁴-1,5-cyclooctadiene)(1-ferrocenyl-1,3-butanedionato-κ²O,O′)iridium(1), [Ir(fca)(cod)],
(13) (η⁴-1,5-cyclooctadiene)(1-ferrocenyl-3-phenyl-1,3-propanedionato-κ²O,O′)iridium(1), [Ir(bfcm)(cod)],
(14) benzoylferrocenoylmethane, (Hbfcm),
(15) ferrocenoylacetone, (Hfca),
(16) (η⁴-1,5-cyclooctadiene)(1-ferrocenyl-1,3-propanedionato-κ²O,O′)iridium(1), [Ir(fch)(cod)].
(17) (η⁴-1,5-cyclooctadiene)(1-ruthenocenyl-1,3-propanedionato-κ²O,O′)rhodium(1), [Rh(RcH)(cod)],
(18) (η⁴-1,5-cyclooctadiene)(1,3-diruthenocenyl-1,3-propanedionato-κ²O,O′)rhodium(1) [Rh(drcm)(cod)],
(19) (η⁴-1,5-cyclooctadiene)(1-ruthenocenyl-3-phenyl-1,3-propanedionato-κ²O,O′)rhodium(1) [Rh(brcm)(cod)],
(20) (η⁴-1,5-cyclooctadiene)(1-ruthenocenyl-1,3-butanedionato-κ²O,O′)rhodium(1), [Rh(rca)(cod)],
(21) (η⁴-1,5-cyclooctadiene)(1-ruthenocenyl-4,4,4-trichloro-1,3-butanedionato-κ²O,O′)rhodium(1), [Rh(rctca)(cod)],
(22) (η⁴-1,5-cyclooctadiene)(1-ruthenocenyl-4,4,4-trifluooro-1,3-butanedionato-κ²O,O′)rhodium(1), [Rh(rctfa)(cod)],
(23) (η⁴-1,5-cyclooctadiene)(1-osmocenyl-1,3-propanedionato-κ²O,O′)rhodium(1), [Rh(OcH)(cod)],
(24) (η⁴-1,5-cyclooctadiene)(1,3-diosmocenyl-1,3-propanedionato-κ²O,O′)rhodium(1) [Rh(docm)(cod)],
(25) (η⁴-1,5-cyclooctadiene)(1-osmocenyl-3-phenyl-1,3-propanedionato-κ²O,O′)rhodium(1) [Rh(bocm)(cod)],
(26) (η⁴-1,5-cyclooctadiene)(1-osmocenyl-1,3-butanedionato-κ²O,O′)rhodium(1), [Rh(oca)(cod)],
(27) (η⁴-1,5-cyclooctadiene)(1-osmocenyl-4,4,4-trichloro-1,3-butanedionato-κ²O,O′)rhodium(1), [Rh(octca)(cod)],
(28) (η⁴-1,5-cyclooctadiene)(1-osmocenyl-4,4,4-trifluooro-1,3-butanedionato-κ²O,O′)rhodium(1), [Rh(octfa)(cod)],
(29) (η⁴-1,5-cyclooctadiene)(1-ferrocenyl-3-ruthenocenyl-1,3-propanedionato-κ²O,O′)rhodium(1) [Rh(fcrcm)(cod)],
(30) (η⁴-1,5-cyclooctadiene)(1-ferrocenyl-3-osmocenyl-1,3-propanedionato-κ²O,O′)rhodium(1) [Rh(fcocm)(cod)],
(31) (β⁴-1,5-cyclooctadiene)(1-osmocenyl-3-ruthenocenyl-1,3-propanedionato-κ²O,O′)rhodium(1) [Rh(ocrcm)(cod)],
(32) (η⁴-1,5-cyclooctadiene)(1,3-pentanedionato-κ²O,O′)rhodium(1) [Rh(acac)(cod)],
(33) 1-ruthenocenyl-1,3-propanedione=HRcH,
(34) 1,3-diruthenocenyl-1,3-propanedione=Hdrcm,
(35) 1-ruthenocenyl-3-phenyl-1,3-propanedione=Hbrcm,
(36) ruthenocenoylacetone=1-ruthenocenyl-1,3-butanedione=Hrca,
(37) ruthenocenoyltrichloroacetone=1-ruthenocenyl-4,4,4-trichloro-1,3-butanedione=Hrctca,
(38) ruthenocenoyltrifluoroacetone=1-ruthenocenyl-4,4,4-trifluoro-1,3-butanedione=Hrctfa,
(39) 1-osmocenyl-1,3-propanedione=HOcH
(40) 1,3-diosmocenyl-1,3-propanedione=Hdocm
(41) 1-osmocenyl-3-phenyl-1,3-propanedione=Hbocm,
(42) osmocenoylacetone=1-osmocenyl-1,3-butanedione=Hoca,
(43) osmocenoyltrichloroacetone=1-osmocenyl-4,4,4-trichloro-1,3-butanedione=Hoctca,
(44) osmocenoyltrifluoroacetone=1-osmocenyl-4,4,4-trifluoro-1,3-butanedione=Hoctfa,
(45) ferrocenoylosmocenoylmethane=1 ferrocenyl-3-osmocenyl-1,3-propanedione=Hfcocm,
(46) osmocenoylruthenocenoylmethane=1-osmocenyl-3-ruthenocenyl-1,3-propanedione=Hrcocm,
(47) ferrocenoylruthenocenoylmethane=1-ferrocenyl-3-ruthenocenyl-1,3-propanedione=Hfcrcm,
(48) (η⁴-1,5-cyclooctadiene)(1-ferrocenyl-3-ruthenocenyl-1,3-propanedionato-κ²O,O′)rhodium(1) [Rh(fcrcm)(cod)],
(49) (η⁴-1,5-cyclooctadiene)(1-ferrocenyl-3-osmocenyl-1,3-propanedionato-κ²O,O′)rhodium(1) [Rh(fcocm)(cod)]

15. A substance or composition for use in a method of sensitizing cells to radiation, the substance or composition including at least one compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which
M is selected from Rh and Ir,
A¹ and A² are the same or different and are selected from cyclic dienes having 6-8 carbons or linear alkenes having 2-7 carbons
B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide.

16. A substance or composition as claimed in claim 15, in which Z₁ and Z₂ are selected from haloalkyl and benzyl.

17. A substance or composition as claimed in claim 15 or claim 16, in which R is selected from CF₃, CCl₃, CH₃, H, Ph (phenyl) and Mc.

18. A substance or composition as claimed in any one of claims 15 to 17 inclusive, in which the halide is selected from F, Cl, Br and I.

19. A substance or composition as claimed in any one of claims 15 to 18 inclusive, in which the pseudohalide is selected from N₃, NCO and SCN.

20. A substance or composition as claimed in any one of claims 15 to 19 inclusive, in which the compound is selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer, [M(β-diketonato)(CO)(COR³)(X)], [M(β-diketonato)(CO)(PR²₃)(R³)(X)] or its acyl isomer, [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

21. A substance or composition as claimed in any one of claims 15 to 20 inclusive, in which the compound is selected from the following compounds 1-50:
(1) ferrocenoylacetaldehyde, (Hfch),
(2) ferrocenoyltrichloroacetone, (Hfctca),
(3) (η⁴-1,5-cyclooctadiene)(1-ferrocenyl-1,3-propanedionato-(4) κ²O,O′)rhodium(1), [Rh(FcH)(cod)],
(4) (η⁴-1,5-cyclooctadiene)(1,3-diferrocenyl-1,3-propanedionato-κ²O,O′)rhodium(1) [Rh(dfcm)(cod)],
(5) (η⁴-1,5-cyclooctadiene)(1-ferrocenyl-3-phenyl-1,3-propanedionato-κ²O,O′)rhodium(1) [Rh(bfcm)(cod)],
(6) (η⁴-1,5-cyclooctadiene)(1-ferrocenyl-1,3-butanedionato-κ²O,O′)rhodium(1), [Rh(fca)(cod)],
(7) (η⁴-1,5-cyclooctadiene)(1-ferrocenyl-4,4,4-trichloro-1,3-butanedionato-κ²O,O′)rhodium(1), [Rh(fctca)(cod)],
(8) (η⁴-1,5-cyclooctadiene)(1-ferrocenyl-4,4,4-trifluooro-1,3-butanedionato-κ²O,O′)rhodium(1), [Rh(fctfa)(cod)],
(9) diferrocenoylmethane, (Hdfcm),
(10) ferrocenoyltrifluoroacetone, (Hfctfa),
(11) (η⁴-1,5-cyclooctadiene)(1-ferrocenyl-4,4,4-trifluoro-1,3-butanedionato-κ²O,O′)iridium(1), [Ir(fctfa)(cod)],
(12) (η⁴-1,5-cyclooctadiene)(1-ferrocenyl-1,3-butanedionato-κ²O,O′)iridium(1), [Ir(fca)(cod)],
(13) (η⁴-1,5-cyclooctadiene)(1-ferrocenyl-3-phenyl-1,3-propanedionato-κ²O,O′)iridium(1), [Ir(bfcm)(cod)],
(14) benzoylferrocenoylmethane, (Hbfcm),
(15) ferrocenoylacetone, (Hfca),
(16) (η⁴-1,5-cyclooctadiene)(1-ferrocenyl-1,3-propanedionato-κ²O,O′)iridium(1), [Ir(fch)(cod)].
(17) (η⁴-1,5-cyclooctadiene)(1-ruthenocenyl-1,3-propanedionato-κ²O,O′)rhodium(1), [Rh(RcH)(cod)],
(18) (η⁴-1,5-cyclooctadiene)(1,3-diruthenocenyl-1,3-propanedionato-κ²O,O′)rhodium(1) [Rh(drcm)(cod)],
(19) (η⁴-1,5-cyclooctadiene)(1-ruthenocenyl-3-phenyl-1,3-propanedionato-κ²O,O′)rhodium(1) [Rh(brcm)(cod)],
(20) (η⁴-1,5-cyclooctadiene)(1-ruthenocenyl-1,3-butanedionato-κ²O,O′)rhodium(1), [Rh(rca)(cod)],
(21) (η⁴-1,5-cyclooctadiene)(1-ruthenocenyl-4,4,4-trichloro-1,3-butanedionato-κ²O,O′)rhodium(1), [Rh(rctca)(cod)],
(22) (η⁴-1,5-cyclooctadiene)(1-ruthenocenyl-4,4,4-trifluooro-1,3-butanedionato-κ²O,O′)rhodium(1), [Rh(rctfa)(cod)],
(23) (η⁴-1,5-cyclooctadiene)(1-osmocenyl-1,3-propanedionato-κ²O,O′)rhodium(1), [Rh(OcH)(cod)],
(24) (η⁴-1,5-cyclooctadiene)(1,3-diosmocenyl-1,3-propanedionato-κ²O,O′)rhodium(1) [Rh(docm)(cod)],
(25) (η⁴-1,5-cyclooctadiene)(1-osmocenyl-3-phenyl-1,3-propanedionato-κ²O,O′)rhodium(1) [Rh(bocm)(cod)],
(26) (η⁴-1,5-cyclooctadiene)(1-osmocenyl-1,3-butanedionato-κ²O,O′)rhodium(1), [Rh(oca)(cod)],
(27) (η⁴-1,5-cyclooctadiene)(1-osmocenyl-4,4,4-trichloro-1,3-butanedionato-κ²O,O′)rhodium(1), [Rh(octca)(cod)],
(28) (η⁴-1,5-cyclooctadiene)(1-osmocenyl-4,4,4-trifluooro-1,3-butanedionato-κ²O,O′)rhodium(1), [Rh(octfa)(cod)],
(29) (η⁴-1,5-cyclooctadiene)(1-ferrocenyl-3-ruthenocenyl-1,3-propanedionato-κ²O,O′)rhodium(1) [Rh(fcrcm)(cod)],
(30) (η⁴-1,5-cyclooctadiene)(1-ferrocenyl-3-osmocenyl-1,3-propanedionato-κ²O,O′)rhodium(1) [Rh(fcocm)(cod)],
(31) (η⁴-1,5-cyclooctadiene)(1-osmocenyl-3-ruthenocenyl-1,3-propanedionato-κ²O,O′)rhodium(1) [Rh(ocrcm)(cod)],
(32) (η⁴-1,5-cyclooctadiene)(1,3-pentanedionato-κ²O,O′)rhodium(1) [Rh(acac)(cod)]
(33) 1-ruthenocenyl-1,3-propanedione=HRcH,
(34) 1,3-diruthenocenyl-1,3-propanedione=Hdrcm,
(35) 1-ruthenocenyl-3-phenyl-1,3-propanedione=Hbrcm,
(36) ruthenocenoylacetone=1-ruthenocenyl-1,3-butanedione=Hrca,
(37) ruthenocenoyltrichloroacetone=1-ruthenocenyl-4,4,4-trichloro-1,3-butanedione=Hrctca,
(38) ruthenocenoyltrifluoroacetone=1-ruthenocenyl-4,4,4-trifluoro-1,3-butanedione=Hrctfa,
(39) 1-osmocenyl-1,3-propanedione=HOcH
(40) 1,3-diosmocenyl-1,3-propanedione=Hdocm
(41) 1-osmocenyl-3-phenyl-1,3-propanedione=Hbocm,
(42) osmocenoylacetone=1-osmocenyl-1,3-butanedione=Hoca,
(43) osmocenoyltrichloroacetone=1-osmocenyl-4,4,4-trichloro-1,3-butanedione=Hoctca,
(44) osmocenoyltrifluoroacetone=1-osmocenyl-4,4,4-trifluoro-1,3-butanedione=Hoctfa,
(45) ferrocenoylosmocenoylmethane=1 ferrocenyl-3-osmocenyl-1,3-propanedione=Hfcocm,
(46) osmocenoylruthenocenoylmethane=1-osmocenyl-3-ruthenocenyl-1,3-propanedione=Hrcocm,
(47) ferrocenoylruthenocenoylmethane=1-ferrocenyl-3-ruthenocenyl-1,3-propanedione=Hfcrcm,
(48) (η⁴-1,5-cyclooctadiene)(1-ferrocenyl-3-ruthenocenyl-1,3-propanedionato-κ²O,O′)rhodium(1) [Rh(fcrcm)(cod)],
(49) (η⁴-1,5-cyclooctadiene)(1-ferrocenyl-3-osmocenyl-1,3-propanedionato-κ²O,O═)rhodium(1) [Rh(fcocm)(cod)]
(50) 1,5-cyclooctadiene)(1,3-pentanedionato-κ²O,O′)rhodium(1) [Rh(acac)(cod)]

22. Use of a substance or composition for the preparation of a medicament for use in sensitizing cells to radiation, the substance or composition including at least one compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which
M is selected from Rh and Ir,
A¹ and A² are the same or different and are selected from cyclic dienes having 6-8 carbons or linear alkenes having 2-7 carbons
B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide.

23. Use as claimed in claim 22, in which Z₁ and Z₂ are selected from haloalkyl and benzyl.

24. Use as claimed in claim 22 or claim 23, in which R is selected from CF₃, CCl₃, CH₃, H, Ph (phenyl) and Mc.

25. Use as claimed in any one of claims 22 to 24 inclusive, in which the halide is selected from F, Cl, Br and I.

26. Use as claimed in any one of claims 22 to 25 inclusive, in which the pseudohalide is selected from N₃, NCO and SCN.

27. Use as claimed in any one of claims 22 to 26 inclusive, in which the compound is selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer, [M(β-diketonato)(CO)(COR³)(X)], [M(β-diketonato)(CO)(PR²₃)(R³)(X)] or its acyl isomer, [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² (is alkyl, phenyl, ferrocenyl and combinations thereof, R³ is alkyl, phenyl or ferrocenyl, and X is a halide or pseudohalide.

28. Use as claimed in any one of claims 22 to 27 inclusive, in which the compound is selected from the following compounds 1-50:
(1) ferrocenoylacetaldehyde, (Hfch),
(2) ferrocenoyltrichloroacetone, (Hfctca),
(3) (η⁴-1,5-cyclooctadiene)(1-ferrocenyl-1,3-propanedionato-(4) κ²O,O′)rhodium(1), [Rh(FcH)(cod)],
(4) (η⁴-1,5-cyclooctadiene)(1,3-diferrocenyl-1,3-propanedionato-κ²O,O′)rhodium(1) [Rh(dfcm)(cod)],
(5) (η⁴-1,5-cyclooctadiene)(1-ferrocenyl-3-phenyl-1,3-propanedionato-κ²O,0O′)rhodium(1) [Rh(bfcm)(cod)],
(6) (η⁴-1,5-cyclooctadiene)(1-ferrocenyl-1,3-butanedionato-κ²O,O′)rhodium(1), [Rh(fca)(cod)],
(7) (η⁴-1,5-cyclooctadiene)(1-ferrocenyl-4,4,4-trichloro-1,3-butanedionato-κ²O,O′)rhodium(1), [Rh(fctca)(cod)],
(8) (η⁴-1,5-cyclooctadiene)(1-ferrocenyl-4,4,4-trifluooro-1,3-butanedionato-κ²O,O′)rhodium(1), [Rh(fctfa)(cod)],
(9) diferrocenoylmethane, (Hdfcm),
(10) ferrocenoyltrifluoroacetone, (Hfctfa),
(11) (η⁴-1,5-cyclooctadiene)(1-ferrocenyl-4,4,4-trifluoro-1,3-butanedionato-κ²O,O′)iridium(1), [Ir(fctfa)(cod)],
(12) (η⁴-1,5-cyclooctadiene)(1-ferrocenyl-1,3-butanedionato-κ²O,O′)iridium(1), [Ir(fca)(cod)],
(13) (η⁴-1,5-cyclooctadiene)(1-ferrocenyl-3-phenyl-1,3-propanedionato-κ²O,O′)iridium(1), [Ir(bfcm)(cod)],
(14) benzoylferrocenoylmethane, (Hbfcm),
(15) ferrocenoylacetone, (Hfca),
(16) (η⁴-1,5-cyclooctadiene)(1-ferrocenyl-1,3-propanedionato-κ²O,O′)iridium(1), [Ir(fch)(cod)].
(17) (η⁴-1,5-cyclooctadiene)(1-ruthenocenyl-1,3-propanedionato-κ²O,O′)rhodium(1), [Rh(RcH)(cod)],
(18) (η⁴-1,5-cyclooctadiene)(1,3-diruthenocenyl-1,3-propanedionato-κ²O,O′)rhodium(1) [Rh(drcm)(cod)],
(19) (η⁴-1,5-cyclooctadiene)(1-ruthenocenyl-3-phenyl-1,3-propanedionato-κ²O,O′)rhodium(1) [Rh(brcm)(cod)],
(20) (η⁴-1,5-cyclooctadiene)(1-ruthenocenyl-1,3-butanedionato-κ²O,O′)rhodium(1), [Rh(rca)(cod)],
(21) (η⁴-1,5-cyclooctadiene)(1-ruthenocenyl-4,4,4-trichloro-1,3-butanedionato-κ²O,O′)rhodium(1), [Rh(rctca)(cod)],
(22) (η⁴-1,5-cyclooctadiene)(1-ruthenocenyl-4,4,4-trifluooro-1,3-butanedionato-κ²O,O′)rhodium(1), [Rh(rctfa)(cod)],
(23) (η⁴-1,5-cyclooctadiene)(1-osmocenyl-1,3-propanedionato-κ²O,O′)rhodium(1), [Rh(OcH)(cod)],
(24) (η⁴-1,5-cyclooctadiene)(1,3-diosmocenyl-1,3-propanedionato-κ²O,O′)rhodium(1) [Rh(docm)(cod)],
(25) (η⁴-1,5-cyclooctadiene)(1-osmocenyl-3-phenyl-1,3-propanedionato-κ²O,O′)rhodium(1) [Rh(bocm)(cod)],
(26) (η⁴-1,5-cyclooctadiene)(1-osmocenyl-1,3-butanedionato-κ²O,O′)rhodium(1), [Rh(oca)(cod)],
(27) (η⁴-1,5-cyclooctadiene)(1-osmocenyl-4,4,4-trichloro-1,3-butanedionato-κ²O,O′)rhodium(1), [Rh(octca)(cod)],
(28) (η⁴-1,5-cyclooctadiene)(1-osmocenyl-4,4,4-trifluooro-1,3-butanedionato-κ²O,O′)rhodium(1), [Rh(octfa)(cod)],
(29) (η⁴-1,5-cyclooctadiene)(1-ferrocenyl-3-ruthenocenyl-1,3-propanedionato-κ²O,O′)rhodium(1) [Rh(fcrcm)(cod)],
(30) (η⁴-1,5-cyclooctadiene)(1-ferrocenyl-3-osmocenyl-1,3-propanedionato-κ²O,O′)rhodium(1) [Rh(fcocm)(cod)],
(31) (η⁴-1,5-cyclooctadiene)(1-osmocenyl-3-ruthenocenyl-1,3-propanedionato-κ²O,O′)rhodium(1) [Rh(ocrcm)(cod)],
(32) (η⁴-1,5-cyclooctadiene)(1,3-pentanedionato-κ²O,O′)rhodium(1) [Rh(acac)(cod)],
(33) 1-ruthenocenyl-1,3-propanedione=HRcH,
(34) 1,3-diruthenocenyl-1,3-propanedione=Hdrcm,
(35) 1-ruthenocenyl-3-phenyl-1,3-propanedione=Hbrcm,
(36) ruthenocenoylacetone=1-ruthenocenyl-1,3-butanedione=Hrca,
(37) ruthenocenoyltrichloroacetone=1-ruthenocenyl-4,4,4-trichloro-1,3-butanedione=Hrctca,
(38) ruthenocenoyltrifluoroacetone=1-ruthenocenyl-4,4,4-trifluoro-1,3-butanedione=Hrctfa,
(39) 1-osmocenyl-1,3-propanedione=HOcH
(40) 1,3-diosmocenyl-1,3-propanedione=Hdocm
(41) 1-osmocenyl-3-phenyl-1,3-propanedione=Hbocm,
(42) osmocenoylacetone=1-osmocenyl-1,3-butanedione=Hoca,
(43) osmocenoyltrichloroacetone=1-osmocenyl-4,4,4-trichloro-1,3-butanedione=Hoctca,
(44) osmocenoyltrifluoroacetone=1-osmocenyl-4,4,4-trifluoro-1,3-butanedione=Hoctfa,
(45) ferrocenoylosmocenoylmethane=1 ferrocenyl-3-osmocenyl-1,3-propanedione=Hfcocm,
(46) osmocenoylruthenocenoylmethane=1-osmocenyl-3-ruthenocenyl-1,3-propanedione=Hrcocm,
(47) ferrocenoylruthenocenoylmethane=1-ferrocenyl-3-ruthenocenyl-1,3-propanedione=Hfcrcm,
(48) (η⁴-1,5-cyclooctadiene)(1-ferrocenyl-3-ruthenocenyl-1,3-propanedionato-κ²O,O′)rhodium(1) [Rh(fcrcm)(cod)],
(49) (η⁴-1,5-cyclooctadiene)(1-ferrocenyl-3-osmocenyl-1,3-propanedionato-κ²O,O′)rhodium(1) [Rh(fcocm)(cod)]
(50) (η⁴-1,5-cyclooctadiene)(1,3-pentanedionato-κ²O,O′)rhodium(1) [Rh(acac)(cod)]

29. A method of sensitizing cells to radiation, the method including the step of exposing the cells before, during or after irradiation to a compound selected from metallocenyl β-diketones of the general formula Mc-CO—CZ₁Z₂-CO—R in which Mc is selected from Fc (ferrocenyl), Rc (ruthenocenyl) and Oc (osmocenyl), R is H, alkyl, haloalkyl or aryl and Z₁ and Z₂ are independently H, alkyl, aryl or substituted alkyl ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which
M is selected from Rh and Ir,
A¹ and A² are the same or different and are selected from cyclic dienes having 6-8 carbons or linear alkenes having 2-7 carbons
B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide, and the method including the step of administering to a person or animal in need of treatment a therapeutically effective dose of the substance or composition.

30. A method as claimed in claim 29, in which Z₁ and Z₂ are selected from haloalkyl and benzyl.

31. A method as claimed in claim 29 or claim 30, in which R is selected from CF₃, CCl₃, CH₃, H, Ph (phenyl) and Mc.

32. A method as claimed in any one of claims 29 to 31 inclusive, in which the halide is selected from F, Cl, Br and I.

33. A method as claimed in any one of claims 29 to 32 inclusive, in which the pseudohalide is selected from N₃, NCO and SCN.

34. A method as claimed in any one of claims 29 to 33 inclusive, in which the compound is selected from the group consisting of [M(β-diketonato)(cod)], [M(β-diketonato)(CO)₂], [M(β-diketonato)(CO)(PR²₃)], [M(β-diketonato){P—(OR²)₃}₂] (M(β-diketonato)(CO)₂(R³)(X)] or its acyl isomer, [M(β-diketonato)(CO)(COR³)(X)], [M(β-diketonato)(CO)(PR²₃)(R³)(X)] or its acyl isomer, [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and ferrocenyl, R² is alkyl, phenyl, ferrocenyl and combinations thereof, R³ is alkyl, phenyl or Mc, and X is a halide or pseudohalide.

35. A method as claimed in any one of claims 29 to 34 inclusive, in which the compound is selected from the following compounds 1-50:
(1) ferrocenoylacetaldehyde, (Hfch),
(2) ferrocenoyltrichloroacetone, (Hfctca),
(3) (η⁴-1,5-cyclooctadiene)(1-ferrocenyl-1,3-propanedionato-(4) κ²O,O′)rhodium(1), [Rh(FcH)(cod)],
(4) (η⁴-1,5-cyclooctadiene)(1,3-diferrocenyl-1,3-propanedionato-κ²O,O′)rhodium(1) [Rh(dfcm)(cod)],
(5) (η⁴-1,5-cyclooctadiene)(1-ferrocenyl-3-phenyl-1,3-propanedionato-κ²O,O′)rhodium(1) [Rh(bfcm)(cod)],
(6) (η⁴-1,5-cyclooctadiene)(1-ferrocenyl-1,3-butanedionato-κ²O,O′)rhodium(1), [Rh(fca)(cod)],
(7) (η⁴-1,5-cyclooctadiene)(1-ferrocenyl-4,4,4-trichloro-1,3-butanedionato-κ²O,O′)rhodium(1), [Rh(fctca)(cod)],
(8) (η⁴-1,5-cyclooctadiene)(1-ferrocenyl-4,4,4-trifluooro-1,3-butanedionato-κ²O,O′)rhodium(1), [Rh(fctfa)(cod)],
(9) diferrocenoylmethane, (Hdfcm),
(10) ferrocenoyltrifluoroacetone, (Hfctfa),
(11) (η⁴-1,5-cyclooctadiene)(1-ferrocenyl-4,4,4-trifluoro-1,3-butanedionato-κ²O,O′)iridium(1), [Ir(fctfa)(cod)],
(12) (η⁴-1,5-cyclooctadiene)(1-ferrocenyl-1,3-butanedionato-κ²O,O′)iridium(1), [Ir(fca)(cod)],
(13) (η⁴-1,5-cyclooctadiene)(1-ferrocenyl-3-phenyl-1,3-propanedionato-κ²O,O′)iridium(1), [Ir(bfcm)(cod)],
(14) benzoylferrocenoylmethane, (Hbfcm),
(15) ferrocenoylacetone, (Hfca),
(16) (η⁴-1,5-cyclooctadiene)(1-ferrocenyl-1,3-propanedionato-κ²O,O′)iridium(1), [Ir(fch)(cod)].
(17) (η⁴-1,5-cyclooctadiene)(1-ruthenocenyl-1,3-propanedionato-κ²O,O′)rhodium(1), [Rh(RcH)(cod)],
(18) (η⁴-1,5-cyclooctadiene)(1,3-diruthenocenyl-1,3-propanedionato-κ²O,O′)rhodium(1) [Rh(drcm)(cod)],
(19) (η⁴-1,5-cyclooctadiene)(1-ruthenocenyl-3-phenyl-1,3-propanedionato-κ²O,O′)rhodium(1) [Rh(brcm)(cod)],
(20) (η⁴-1,5-cyclooctadiene)(1-ruthenocenyl-1,3-butanedionato-κ²O,O′)rhodium(1), [Rh(rca)(cod)],
(21) (η⁴-1,5-cyclooctadiene)(1-ruthenocenyl-4,4,4-trichloro-1,3-butanedionato-κ²O,O′)rhodium(1), [Rh(rctca)(cod)],
(22) (η⁴-1,5-cyclooctadiene)(1-ruthenocenyl-4,4,4-trifluooro-1,3-butanedionato-κ²O,O′)rhodium(1), [Rh(rctfa)(cod)],
(23) (ρ⁴-1,5-cyclooctadiene)(1-osmocenyl-1,3-propanedionato-κ²O,O′)rhodium(1), [Rh(OcH)(cod)],
(24) (η⁴-1,5-cyclooctadiene)(1,3-diosmocenyl-1,3-propanedionato-κ²O,O′)rhodium(1) [Rh(docm)(cod)],
(25) (η⁴-1,5-cyclooctadiene)(1-osmocenyl-3-phenyl-1,3-propanedionato-κ²O,O′)rhodium(1) [Rh(bocm)(cod)],
(26) (η⁴-1,5-cyclooctadiene)(1-osmocenyl-1,3-butanedionato-κ²O,O′)rhodium(1), [Rh(oca)(cod)],
(27) (η⁴-1,5-cyclooctadiene)(1-osmocenyl-4,4,4-trichloro-1,3-butanedionato-κ²O,O′)rhodium(1), [Rh(octca)(cod)],
(28) (η⁴-1,5-cyclooctadiene)(1-osmocenyl-4,4,4-trifluooro-1,3-butanedionato-κ²O,O′)rhodium(1), [Rh(octfa)(cod)],
(29) (η⁴-1,5-cyclooctadiene)(1-ferrocenyl-3-ruthenocenyl-1,3-propanedionato-κ²O,O′)rhodium(1) [Rh(fcrcm)(cod)],
(30) (η⁴-1,5-cyclooctadiene)(1-ferrocenyl-3-osmocenyl-1,3-propanedionato-κ²O,O′)rhodium(1) [Rh(fcocm)(cod)],
(31) (η⁴-1,5-cyclooctadiene)(1-osmocenyl-3-ruthenocenyl-1,3-propanedionato-κ²O,O′)rhodium(1) [Rh(ocrcm)(cod)],
(32) (η⁴-1,5-cyclooctadiene)(1,3-pentanedionato-κ²O,O′)rhodium(1) [Rh(acac)(cod)]
(33) 1-ruthenocenyl-1,3-propanedione=HRcH,
(34) 1,3-diruthenocenyl-1,3-propanedione=Hdrcm,
(35) 1-ruthenocenyl-3-phenyl-1,3-propanedione=Hbrcm,
(36) ruthenocenoylacetone=1-ruthenocenyl-1,3-butanedione=Hrca,
(37) ruthenocenoyltrichloroacetone=1-ruthenocenyl-4,4,4-trichloro-1,3-butanedione=Hrctca,
(38) ruthenocenoyltrifluoroacetone=1-ruthenocenyl-4,4,4-trifluoro-1,3-butanedione=Hrctfa,
(39) 1-osmocenyl-1,3-propanedione=HOcH
(40) 1,3-diosmocenyl-1,3-propanedione=Hdocm
(41) 1-osmocenyl-3-phenyl-1,3-propanedione=Hbocm,
(42) osmocenoylacetone=1-osmocenyl-1,3-butanedione=Hoca,
(43) osmocenoyltrichloroacetone=1-osmocenyl-4,4,4-trichloro-1,3-butanedione=Hoctca,
(44) osmocenoyltrifluoroacetone=1-osmocenyl-4,4,4-trifluoro-1,3-butanedione=Hoctfa,
(45) ferrocenoylosmocenoylmethane=1 ferrocenyl-3-osmocenyl-1,3-propanedione=Hfcocm,
(46) osmocenoylruthenocenoylmethane=1-osmocenyl-3-ruthenocenyl-1,3-propanedione=Hrcocm,
(47) ferrocenoylruthenocenoylmethane=1-ferrocenyl-3-ruthenocenyl-1,3-propanedione=Hfcrcm,
(48) (η⁴-1,5-cyclooctadiene)(1-ferrocenyl-3-ruthenocenyl-1,3-propanedionato-κ²O,O′)rhodium(1) [Rh(fcrcm)(cod)],
(49) (η⁴-1,5-cyclooctadiene)(1-ferrocenyl-3-osmocenyl-1,3-propanedionato-κ²O,O′)rhodium(1) [Rh(fcocm)(cod)]
(50) (η⁴-1,5-cyclooctadiene)(1,3-pentanedionato-κ²O,O′)rhodium(1) [Rh(acac)(cod)].

36. A metallocenyl β-diketone of the general formula Mc-CO—C-Z₁Z₂-CO—R in which Mc is selected from Fc (ferrocenyl), Rc (ruthenocenyl) and Oc (osmocenyl), R is H, alkyl or aryl and Z₁ and Z₂ are independently H, alkyl, aryl or substituted alkyl, ferrocenyl, the enol forms of the β-diketones and metal complexes of the β-diketones of the general formula M(β-diketonato)A¹, M(β-diketonato)A¹A², M(β-diketonato)A¹B¹B², M(β-diketonato)B¹B² and M(β-diketonato)B¹B²B³B⁴ in which
M is selected from Rh and Ir,
A¹ and A² are the same or different and are selected from cyclic dienes having 6-8 carbons, or linear alkenes having 2-7 carbons
B¹, B², B³ and B⁴ are the same or different and are selected from CO, P(R¹R²R³), P(OR¹)(OR²)(OR³), R⁴ and X in which R¹, R², R³ and R⁴ are the same or different and are independently selected from alkyl, phenyl and ferrocenyl, and X is a halide or a pseudohalide, with the proviso that the compound may not be
(1) ferrocenoyltrichloroacetone, (Hfctca),
(2) (η¹-1,5-cyclooctadiene)(1,3-diferrocenyl-1,3-propanedionato-κ²O,O′)rhodium(1) [Rh(dfcm)(cod)],
(3) (η⁴-1,5-cyclooctadiene)(1-ferrocenyl-3-phenyl-1,3-propanedionato-κ²O,O′)rhodium(1) [Rh(bfcm)(cod)],
(4) (η⁴-1,5-cyclooctadiene)(1-ferrocenyl-1,3-butanedionato-κ²O,O′)rhodium(1), [Rh(fca)(cod)],
(5) (η⁴-1,5-cyclooctadiene)(1-ferrocenyl-4,4,4-trichloro-1,3-butanedionato-κ²O,O′)rhodium(1), [Rh(fctca)(cod)],
(6) (η⁴-1,5-cyclooctadiene)(1-ferrocenyl-4,4,4-trifluooro-1,3-butanedionato-κ²O,O′)rhodium(1), [Rh(fctfa)(cod)],
(7) diferrocenoylmethane, (Hdfcm),
(8) ferrocenoyltrifluoroacetone, (Hfctfa),
(9) benzoylferrocenoylmethane, (Hbfcm),
(10) ferrocenoylacetone, (Hfca),
(11) (η⁴-1,5-cyclooctadiene)(1,3-pentanedionato-κ²O,O′)rhodium(1) [Rh(acac)(cod)],

37. A metallocenyl β-diketone as claimed in claim 36, in which Z₁ and Z₂ are be selected from haloalkyl and benzyl.

38. A metallocenyl β-diketone as claimed in claim 36 or claim 37, in which R is selected from CF₃, CCl₃, CH₃, H, Ph (phenyl) and Mc.

39. A metallocenyl β-diketone as claimed in any one of claims 36 to 38 inclusive, in which the halide is F, Cl, Br or I.

40. A metallocenyl β-diketone as claimed in any one of claims 36 to 39 inclusive, in which the pseudohalide is N₃, NCO or SCN.

41. A metallocenyl β-diketone as claimed in any one of claims 36 to 40 inclusive, which is selected from the group consisting of [M(β-diketonato)(cod)], [M(β-diketonato)(CO)₂], [M(β-diketonato)(CO)(PR²₃)], [M(β-diketonato){P—(OR²)₃}₂] [M(β-diketonato)(CO)₂(R³)(X)] or its acyl isomer [M(β-diketonato)(CO)(COR³)(X)], [M(β-diketonato)(CO)(PR²₃)(R³)(X)] or its acyl isomer [M(β-diketonato)(PR²₃)(COR³)(X)], [M(β-diketonato){P—(OR²)₃}₂(R³)(X)] and [M(β-diketonato)(cod)(R³)(X)], in which M is Rh or Ir, cod is 1,5-cyclooctadiene, (β-diketonato) is (McCOCHCOR) in which R is selected from CF₃, CCl₃, CH₃, H, phenyl and Mc, R² is alkyl, phenyl, ferrocenyl and combinations thereof, R³ is alkyl, phenyl or ferrocenyl, and X is a halide or pseudohalide.

42. A metallocenyl β-diketone as claimed in any one of claims 36 to 41 inclusive, which is selected from the group consisting of:
(1) ferrocenoylacetaldehyde, (Hfch),
(2) (η⁴-1,5-cyclooctadiene)(1-ferrocenyl-1,3-propanedionato-
(3) diferrocenoylmethane, (Hdfcm),
(4) ferrocenoyltrifluoroacetone, (Hfctfa),
(5) (η⁴-1,5-cyclooctadiene)(1-ferrocenyl-4,4,4-trifluoro-1,3-butanedionato-κ²O,O′)iridium(1), [Ir(fctfa)(cod)],
(6) (η⁴-1,5-cyclooctadiene)(1-ferrocenyl-1,3-butanedionato-κ²O,O′)iridium(1), [Ir(fca)(cod)],
(7) (η⁴-1,5-cyclooctadiene)(1-ferrocenyl-3-phenyl-1,3-propanedionato-κ²O,O′)iridium(1), [Ir(bfcm)(cod)],
(8) (η⁴-1,5-cyclooctadiene)(1-ferrocenyl-1,3-propanedionato-κ²O,O′)iridium(1), [Ir(fch)(cod)].
(9) (η⁴-1,5-cyclooctadiene)(1-ruthenocenyl-1,3-propanedionato-κ²O,O′) rhodium(1), [Rh(RcH) (cod)],
(10) (η⁴-1,5-cyclooctadiene)(1,3-diruthenocenyl-1,3-propanedionato-κ²O,O′)rhodium(1) [Rh(drcm)(cod)],
(11) (η⁴-1,5-cyclooctadiene)(1-ruthenocenyl-3-phenyl-1,3-propanedionato-κ²O,O′)rhodium( 1) [Rh(brcm)(cod)],
(1 2) (η⁴-1,5-cyclooctadiene)(1-ruthenocenyl-1,3-butanedionato-κ²O,O′)rhodium(1), [Rh(rca)(cod)],
(13) (η⁴-1,5-cyclooctadiene)(1-ruthenocenyl-4,4,4-trichloro-1,3-butanedionato-κ²O,O′)rhodium(1), [Rh(rctca)(cod)],
(14) (η⁴-1,5-cyclooctadiene)(1-ruthenocenyl-4,4,4-trifluooro-1,3-butanedionato-κ²O,O′)rhodium(1), [Rh(rctfa)(cod)],
(15) (η⁴-1,5-cyclooctadiene)(1-osmocenyl-1,3-propanedionato-κ²O,O′)rhodium(1), [Rh(OcH)(cod)],
(16) (η⁴-1,5-cyclooctadiene)(1,3-diosmocenyl-1,3-propanedionato-κ²O,O′)rhodium(1) [Rh(docm)(cod)],
(17) (η⁴-1,5-cyclooctadiene)(1-osmocenyl-3-phenyl-1,3-propanedionato-κ²O,O′) rhodium(1) [Rh(bocm)(cod)],
(18) (η⁴-1,5-cyclooctadiene)(1-osmocenyl-1,3-butanedionato-κ²O,O′)rhodium(1), [Rh(oca)(cod)],
(19) (η⁴-1,5-cyclooctadiene)(1-osmocenyl-4,4,4-trichloro-1,3-butanedionato-κ²O,O′)rhodium(1), [Rh(octca)(cod)],
(20) (η⁴-1,5-cyclooctadiene)(1-osmocenyl-4,4,4-trifluooro-1,3-butanedionato-κ²O,O′)rhodium(1), [Rh(octfa)(cod)],
(21) (η⁴-1,5-cyclooctadiene)(1-ferrocenyl-3-ruthenocenyl-1,3-propanedionato-κ²O,O′)rhodium(1) [Rh(fcrcm)(cod)],
(22) (η⁴-1,5-cyclooctadiene)(1-ferrocenyl-3-osmocenyl-1,3-propanedionato-κ²O,O′)rhodium(1) [Rh(fcocm)(cod)],
(23) (η⁴-1,5-cyclooctadiene)(1-osmocenyl-3-ruthenocenyl-1,3-propanedionato-κ²O,O′)rhodium(1) [Rh(ocrcm)(cod)],
(24) (η⁴-1,5-cyclooctadiene)(1,3-pentanedionato-κ²O,O′)rhodium(1) [Rh(acac)(cod)],
(25) 1-ruthenocenyl-1,3-propanedione=HRcH,
(26) 1,3-diruthenocenyl-1,3-propanedione=Hdrcm,
(27) 1-ruthenocenyl-3-phenyl-1,3-propanedione=Hbrcm,
(28) ruthenocenoylacetone=1-ruthenocenyl-1,3-butanedione=Hrca,
(29) ruthenocenoyltrichloroacetone=1-ruthenocenyl-4,4,4-trichloro-1,3-butanedione=Hrctca,
(30) ruthenocenoyltrifluoroacetone=1-ruthenocenyl-4,4,4-trifluoro-1,3-butanedione=Hrctfa,
(31) 1-osmocenyl-1,3-propanedione=HOcH
(32) 1,3-diosmocenyl-1,3-propanedione=Hdocm
(33) 1-osmocenyl-3-phenyl-1,3-propanedione=Hbocm,
(34) osmocenoylacetone=1-osmocenyl-1,3-butanedione=Hoca,
(35) osmocenoyltrichloroacetone=1-osmocenyl-4,4,4-trichloro-1,3-butanedione=Hoctca,
(36) osmocenoyltrifluoroacetone=1-osmocenyl-4,4,4-trifluoro-1,3-butanedione=Hoctfa,
(37) ferrocenoylosmocenoylmethane=1 ferrocenyl-3-osmocenyl-1,3-propanedione=Hfcocm,
(38) osmocenoylruthenocenoylmethane=1-osmocenyl-3-ruthenocenyl-1,3-propanedione=Hrcocm,
(39) ferrocenoylruthenocenoylmethane=1-ferrocenyl-3-ruthenocenyl-1,3-propanedione=Hfcrcm,
(40) (η⁴-1,5-cyclooctadiene)(1-ferrocenyl-3-ruthenocenyl-1,3-propanedionato-κ²O,O′)rhodium(1) [Rh(fcrcm)(cod)],
(41) (η⁴-1,5-cyclooctadiene)(1-ferrocenyl-3-osmocenyl-1,3-propanedionato-κ²O,O′)rhodium(1) [Rh(fcocm)(cod)]

43. A substance or composition as claimed in claim 1 or claim 15 or claim 18 substantially as herein described and illustrated.

44. Use as claimed in claim 8 or claim 22 substantially as herein described.

45. A method as claimed in claim 29 substantially as herein described and illustrated.

46. A metallocenyl β-diketone as claimed in claim 36, substantially as herein described and illustrated.

47. A new substance or composition, a new use, a new method or a new metallocenyl β-diketone substantially as herein described.
